(19) United States
(12) Patent Application Publication
(10) Pub. No.: US 2025/0042909 A1
(43) Pub. Date: Feb. 6, 2025

(54) POLYMORPH FORMS OF A 5H-PYRROLO[2,3-b]PYRAZINE DERIVATIVE, METHODS OF PREPARATION, AND USES THEREFORE

(71) Applicant: BeiGene, Ltd., Camana Bay (KY)

(72) Inventors: Hui Chen, Suzhou (CN); Sanjia Xu, Beijing (CN); Jing Li, Beijing (CN); Zhengming Du, Suzhou (CN); Zhiwei Wang, Beijing (CN)

(73) Assignee: BeiGene, Ltd., Camana Bay (KY)

(21) Appl. No.: 18/924,058

(22) Filed: Oct. 23, 2024

Related U.S. Application Data
(63) Continuation of application No. PCT/CN23/90248, filed on Apr. 24, 2023.

(30) Foreign Application Priority Data
Apr. 24, 2022 (WO) PCT/CN22/88761

(51) Int. Cl. C07D 487/04; A61K 31/4985

(52) U.S. Cl. CPC C07D 487/04; A61K 31/4985

(57) ABSTRACT

The present invention relates to salts of a HPK1 inhibitor (referred to as “Compound A” hereinafter), preferably citrate, and the crystalline forms thereof. The present invention also relates to the process of preparation and uses of the salts and crystalline forms of Compound A.

### CROSS-REFERENCE TO RELATED APPLICATIONS

This application is a continuation of International Application No. PCT/CN2023/090248, filed Apr. 24, 2023, which claims priority to International Application No. PCT/CN2022/088761, filed Apr. 24, 2022. The disclosures of the aforementioned applications are incorporated herein by reference in their entireties.

### FIELD OF THE INVENTION

The present invention relates to polymorphs of a hematopoietic progenitor kinase 1 (HPK1) inhibitor (referred to as “Compound A” hereinafter), preferably polymorphs of the citrate salt of the compound. The present invention also relates to the process of preparation and uses of the salts and crystalline forms of Compound A.

### BACKGROUND OF THE INVENTION

The compound 4-[2-(2,8-dimethyl-1,2,3,4-tetrahydroisoquinolin-6-yl)-5H-pyrrolo[2,3-b]pyrazin-7-yl]-N,N,2-trimethylbenzamide (Compound A) is presented by the formula below:

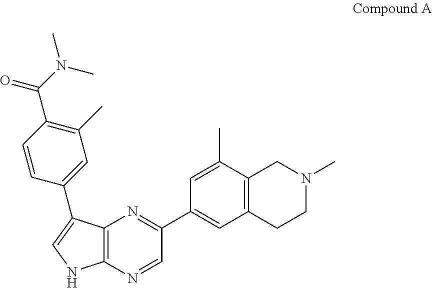 [SMILES: Cc1cc(-c2c[nH]c3ncc(-c4cc(C)c5c(c4)CCN(C)C5)nc23)ccc1C(=O)N(C)C]

WO2021000925 disclosed a series of compounds including Compound A. Compound A was described as an HPK1 inhibitor useful in the treatment of various diseases including cancer.

In order to be manufactured into pharmaceutical products, it is strictly required that the active ingredient must have high purity and stability. Particularly, in order to maintain high stability in a longer shelf period, the active ingredient must have low hygroscopicity so that the influence on the quality by moisture can be avoided. Thus, Compound A needs to be manufactured in crystalline either as free forms or salts to pursue improved properties.

For orally administered solid formulations comprising the desired active ingredient, the active ingredient needs to have the desired bioavailability so that the active ingredient could be absorbed into the blood circulation of the body as much as possible. However, the relationship between the bioavailability and the specific salt is unknown in the art, and a new salt of Compound A with sufficient bioavailability is highly desired.

HPK1 is a member of MAP4Ks family, which includes MAP4K1/HPK1, MAP4K2/GCK, MAP4K3/GLK, MAP4K4/HGK, MAP4K5/KHS, MAP4K6/MINK [Hu, M. C., et al., Genes Dev, 1996. 10: pp. 2251-64]. HPK1 regulates diverse functions ofvarious immune cells and its kinase activity has been shown to be induced upon activation of T cell receptors (TCR) [Liou J., et al., Immunity, 2000. 12 (4): pp. 399-408], B cell receptors (BCR) [Liou J, et al., Immunity, 2000. 12 (4): pp. 399-408], transforming growth factor receptor (TGF-βR) [Wang, W., et al., J Biol Chem, 1997 272 (36): pp. 22771-5; Zhou, G., et al., J Biol Chem, 1999. 274 (19): pp. 13133-8], and Gs-coupled PGE2 receptors (EP2 and EP4) [Ikegami, R., et al., J Immunol, 2001. 166 (7): pp. 4689-96]. Overexpression of HPK1 suppresses TCR-induced activation of AP-1-dependent gene transcription in a kinase-dependent manner, suggesting that HPK1 is required to inhibit the Erk MAPK pathway [Liou J., et al., Immunity, 2000. 12 (4): pp. 399-408] and this blockage is thought to be the inhibitory mechanism that negatively regulates TCR-induced IL-2 gene transcription [S. Sawasdikosol., et al., Immunol Res, 2012. 54: pp. 262-265].

In vitro HPK1−/− T cells have a lower TCR activation threshold, proliferate robustly, produce enhanced amounts of Th1 cytokines, the HPK1−/− mice experience more severe autoimmune symptoms [S. Sawasdikosol., et al., Immunol Res, 2012. 54: pp. 262-265]. In human, HPK1 was downregulated in peripheral blood mononuclear cells of psoriatic arthritis patients or T cells of systemic lupus erythematosus (SLE) patients [Batliwalla F M, et al., Mol Med, 2005. 11 (1-12): pp. 21-9], which indicated that attenuation of HPK1 activity may contribute to autoimmunity in patients. Furthermore, HPK1 may also control anti-tumor immunity via T cell-dependent mechanisms. In the PGE2-producing Lewis lung carcinoma tumor model, the tumors developed more slowly in HPK1 knockout mice as compared to wild-type mice [US patent application No. 2007 0087988]. HPK1 deficient T cells were more effective in controlling tumor growth and metastasis than wild-type T cells [Alzabin, S., et al., Cancer Immunol Immunother, 2010. 59 (3): pp. 419-29]. Similarly, BMDCs from HPK1 knockout mice were more efficient to mount a T cell response to eradicate Lewis lung carcinoma as compared to wild-type BMDCs [Alzabin, S., et al., J Immunol, 2009. 182 (10): pp. 6187-94]. In all, HPK1 may be a good target for enhancing antitumor immunity.

Therefore, it remains the need for the discovery of new solid forms of Compound A or the salts thereof to meet the above pharmaceutical formulation requirements.

### SUMMARY OF THE INVENTION

The present application discloses an invention to address the foregoing challenges and needs by providing stable salts of Compound A, and especially a citrate salt of Compound A, which shows the desired crystallinity and improved bioavailability suitable for pharmaceutical formulation.

In addition, the inventors have found that among different salts of Compound A, the citrate salt of Compound A shows unpredictable high bioavailability, which makes the citrate salt of Compound A suitable for pharmaceutical formulation.

Surprisingly, salts of Compound A, preferably citrate salt of Compound A, even more preferably the crystalline of citrate is a solid with very low hygroscopicity which results in a good flowability for industrial production. The salts of Compound A, preferably citrate salt of Compound A, even more preferably the crystalline of citrate can be used in the large-scale production of formulation process without the hygroscopicity problem.

Even more surprisingly, the citrate salt type A showed an excellent long-term stability during the 3-month experiment. From the current data, we also could expect that citrate salt type A should have a very good long-term stability, such as 6-month long-term stability, 12-month long-term stability, 24-month long-term stability and 36-month long-term stability.

Before the filing date of the instant application, the inventors of the instant application have unexpectedly found that only citric acid can form crystalline forms with the desired crystallinity, high stability, low hygroscopicity with Compound A.

Aspect 1. A pharmaceutically acceptable salt of 4-[2-(2,8-dimethyl-1,2,3,4-tetrahydroisoquinolin-6-yl)-5H-pyrrolo[2,3-b]pyrazin-7-yl]-N,N,2-trimethylbenzamide, wherein said pharmaceutically acceptable salts are inorganic salt(s) or organic salt(s).

Aspect 2. The salt according to Aspect 1, which is in solid-state.

Aspect 3. The salt according to Aspect 1 or 2, wherein the salt is an inorganic salt selected from hydrochloride, sulphate, phosphate, hydrobromide and/or nitrate; or is an organic salt selected from fumarate, tartrate (L-tartrate, D-tartrate or DL-tartrate), laurate, stearate, gentisate, nicotinate, aspartate (L-aspartate), succinate, adipate, malate (L-malate), citrate, maleate, glycolate, gluconate (D-gluconate), lactate (L-lactate), acetate, benzene sulfonate, methanesulfonate, mesylate, benzoate, naphthalene sulfonate, and/or oxalate;
preferably, the salt is selected from hydrochloride, sulphate, phosphate, hydrobromide, fumarate, tartrate (L-tartrate, D-tartrate or DL-tartrate), aspartate (L-aspartate), succinate, malate (L-malate), citrate, maleate, methanesulfonate or mesylate;
more preferably, the salt is selected from L-malate, citrate or succinate;
even more preferably, the salt is citrate.

Aspect 4. The salt according to Aspect 3, wherein the salt is a compound of Formula (I):

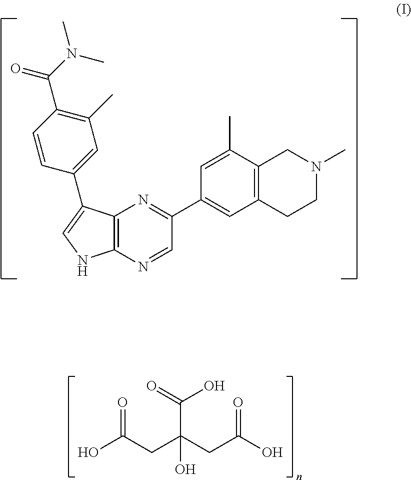

wherein n is a number from about 0.2 to about 2.0.

Aspect 5. The salt according to Aspect 4, wherein n is a number about 0.3 to about 1.5; preferably n is a number selected from the group consisting of 0.3±0.1, 0.5±0.1, 0.7±0.1, 1.0±0.1 and 1.5±0.1;
preferably, n is a number selected from 0.3±0.05, 0.5±0.05, 0.6±0.05, 0.7±0.05, 0.8±0.05, 1.0±0.05, 1.1±0.05 and 1.5±0.05;
more preferably, n is 0.25˜0.35, 0.55˜0.65, 0.65˜0.75, 0.75˜0.85, 0.85˜0.95, 0.95˜1.05, 1.05˜1.15 or 1.45˜1.55;
even more preferably, n is about 0.3, 0.33, 0.40, 0.44, 0.45, 0.50, 0.55, 0.56, 0.60, 0.63, 0.65, 0.66, 0.67, 0.70, 0.75, 0.76, 0.80, 0.85, 0.90, 0.95, 0.96, 0.97, 0.98, 0.99, 1.0, 1.01, 1.02, 1.03, 1.04, 1.05, 1.06, 1.1, 1.45, 1.5, 1.55, 1.67, 1.8, 1.90, 1.95, 2.0.

Aspect 6. The salt according to Aspect 3, wherein the salt is L-malate.

Aspect 7. The salt according to Aspect 6, wherein the salt is a compound of Formula (II):

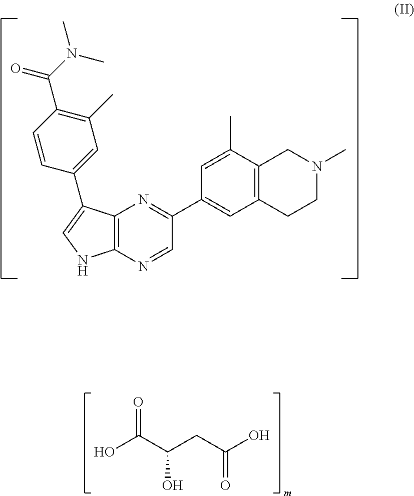

wherein m is a number from about 0.5 to about 2.0.

Aspect 8. The salt according to Aspect 7, wherein m is a number about 0.5 to about 1.5;
preferably m is a number selected from the group consisting of 0.5±0.1, 0.7±0.1, 1.0±0.1 and 1.5±0.1;
more preferably, m is a number selected from 0.5±0.05, 0.6±0.05, 0.7±0.05, 0.8±0.05, 0.9±0.05, 1.0±0.05, 1.1±0.05, 1.2±0.05 and 1.5±0.05;
even more preferably, m is 0.95˜1.05, 1.05˜1.15, 1.15˜1.25 or 1.45˜1.55;
even more preferably, m is about 0.45, 0.50, 0.55, 0.95, 0.98, 0.99, 1.0, 1.01, 1.02, 1.05, 1.06, 1.07, 1.08, 1.09, 1.10, 1.11, 1.12, 1.13, 1.14, 1.15, 1.16, 1.17, 1.18, 1.19, 1.20, 1.3, 1.4, 1.45 or 1.5.

Aspect 9. The salt according to Aspect 3, wherein the salt is succinate.

Aspect 10. The salt according to Aspect 9, wherein the salt is a compound of Formula (III):

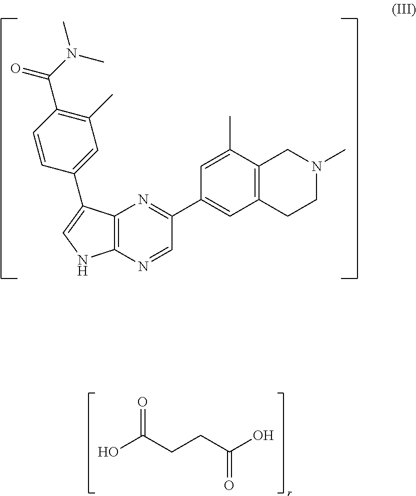

wherein r is a number from about 0.2 to about 2.0.

Aspect 11. The salt according to Aspect 10, wherein r is a number about 0.5 to about 1.5;
preferably r is a number selected from the group consisting 0.5±0.1, 0.7±0.1, 1.0±0.1 and 1.5±0.1;
more preferably, r is a number selected from 0.5±0.05, 0.6±0.05, 0.7±0.05, 0.8±0.05, 0.9±0.05, 1.0±0.05, 1.1±0.05, 1.2±0.05 and 1.5±0.05;
even more preferably, r is 0.95˜1.05, 1.05˜1.15, 1.15˜1.25 or 1.45˜1.55;
even more preferably, r is 0.45, 0.50, 0.55, 0.95, 0.98, 0.99, 1.0, 1.01, 1.02, 1.05, 1.06, 1.07, 1.08, 1.09, 1.10, 1.11, 1.12, 1.13, 1.14, 1.15, 1.16, 1.17, 1.18, 1.19, 1.20, 1.3, 1.4, 1.45 or 1.5.

Aspect 12. A pharmaceutical composition comprising a therapeutically effective amount of the salts according to any one of Aspects 1-11, and optionally one or more pharmaceutically acceptable carrier(s).

Aspect 13. A method for treating or preventing a disorder or a disease selected from inflammatory disorder, autoimmune disease, or cancer, comprising administering a subject in need thereof a therapeutically effective amount of the salts according to any one of Aspects 1-11, or the pharmaceutical composition of Aspect 12.

Aspect 14. A crystalline form of Formula IV

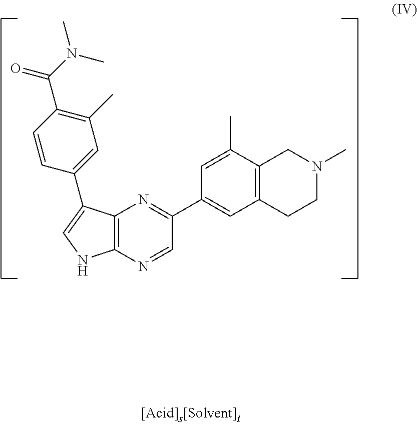

wherein [Acid] is selected from the group consisting of organic acids and inorganic acids;
[Solvent] is selected from H₂O or organic solvents;
s is a number from about 0.0 to about 5.0;
t is a number from about 0.0 to about 5.0.

Aspect 15. A crystalline form of Aspect 14, wherein [Acid] is selected from the group consisting of hydrochloric acid, sulfuric acid, phosphoric acid, hydrobromic acid, nitric acid, fumaric acid, L-tartaric acid, D-tartaric acid, DL-tartaric acid, lauric acid, stearic acid, gentistic acid, nicotinic acid, aspartic acid, succinic acid, adipic acid, malic acid (L-malic acid), citric acid, maleic acid, ascorbic acid (L-ascorbic acid), glycolic acid, gluconic acid (D-gluconic acid), lactic acid (L-lactic acid), acetic acid, benzenesulfonic acid, methanesulfonic acid, benzoic acid, naphthalene sulfonic acid, and/or oxalic acid;
preferably [Acid] is selected from hydrochloric acid, sulfuric acid, phosphoric acid, hydrobromic acid, fumaric acid, tartaric acid (L-tartaric acid or D-tartaric acid), aspartic acid (L-aspartic acid), succinic acid, malic acid (L-malic acid), citric acid, maleic acid, methanesulfonic acid;
more preferably [Acid] is selected from L-malic acid, citric acid, succinic acid;
even more preferably [Acid] is selected from citric acid.

Aspect 16. A crystalline form of any one of Aspects 14-15, wherein s is a number about 0 to about 1.5;
preferably s is a number selected from the group consisting 0.3±0.1, 0.5±0.1, 0.7±0.1, 1.0±0.1 and 1.5±0.1;
more preferably s is a number selected from the group consisting of 0.3±0.05, 0.5±0.05, 0.6±0.05, 0.7±0.05, 0.8±0.05, 1.0±0.05, 1.1±0.05, 1.2±0.05 and 1.5±0.05;
even more preferably, s is a number selected from the group consisting of 0.25˜0.35, 0.55˜0.65, 0.65˜0.75, 0.75˜0.85, 0.85˜0.95, 0.95˜1.05, 1.05˜1.15, 1.15˜1.25 or 1.45˜1.55;
even more preferably, s is 0.55˜0.65, 0.65˜0.75, 0.75˜0.85, 0.85˜0.95, 0.95˜1.05, 1.05˜1.15 or 1.45˜1.55; even more preferably, s is 0.3, 0.33, 0.40, 0.44, 0.45, 0.50, 0.55, 0.56, 0.60, 0.63, 0.65, 0.66, 0.67, 0.70, 0.75, 0.76, 0.80, 0.85, 0.90, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.20, 1.3, 1.4, 1.45, 1.5, 1.55, 1.67, 1.8, 1.90, 1.95, or 2.0.

Aspect 17. A crystalline form of Aspect 14, wherein [Solvent] is selected from the group consisting of inorganic solvents selected from H₂O, MeOH, EtOH, n-PrOH, i-PrOH, 1-Butanol, sec-BuOH, tert-BuOH CF₃CH₂OH, acetone, toluene, THF, MeOAc, EtOAc, PrOAc, dioxane chloroform, DCM, butanone and MeCN or a combination of any of the foregoing;
preferably, [Solvent] is selected from the group consisting of inorganic solvents selected from H₂O, MeOH, EtOH, n-PrOH, i-PrOH, 1-BuOH, sec-BuOH, tert-BuOH, CF₃CH₂OH, acetone, toluene, THF, MeOAc, EtOAc, PrOAc, dioxane, chloroform, DCM, butanone, MeCN, (H₂O and EtOH), (H₂O and acetone) or (H₂O and MeCN);
even more preferably, solvents selected from H₂O, MeOH, EtOH, n-PrOH, i-PrOH, CF₃CH₂OH or (H₂O and EtOH) or (H₂O and MeOH) or (H₂O and i-PrOH) or (H₂O and n-PrOH) or (H₂O, n-PrOH and i-PrOH) or any combinations thereof.

Aspect 18. A crystalline form of any one of Aspects 14 and 17, wherein t is a number about 0 to about 3;
preferably t is a number selected from the group consisting of 0, 0.5±0.2, 1.0±0.2, 1.5±0.2, 2.0±0.2, 2.5±0.2, 3.0±0.2;
more preferably, t is 0˜0.13, 0.13˜0.25, 0.25˜0.5, 0.5˜0.67, 0.67˜0.75, 0.75˜1, 1˜1.5, 1.5˜2, 2˜2.5, 2.5˜3;
even more preferably, t is about 0, 0.1, 0.14, 0.17, 0.2, 0.25, 0.3, 0.4, 0.45, 0.5, 0.50, 0.55, 0.57, 0.6, 0.65, 0.7, 0.8, 0.9, 0.95, 0.98, 1.02, 1.05, 1.08, 1.09, 1.0, 1.1, 1.11, 1.12, 1.2, 1.3, 1.4, 1.45, 1.5, 1.55, 1.6, 1.7, 1.8, 1.9, 2.0, 2.1, 2.2, 2.25, 2.3, 2.31, 2.4, 2.5, 2.6, 2.7, 2.8, 2.9, or 3.0.

Aspect 19. A crystalline form of Aspects 14, wherein the crystalline form is free base and the crystalline form is Formula Va

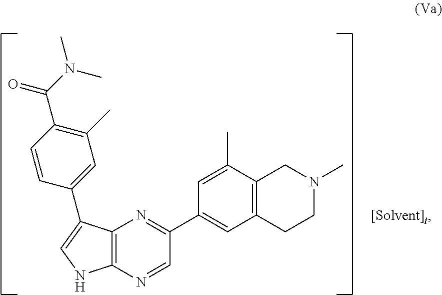

wherein [solvent] and t are as defined as Aspect 14;
preferably, the crystalline form is Formula Vb, Vc, Vd, Ve or Vf:

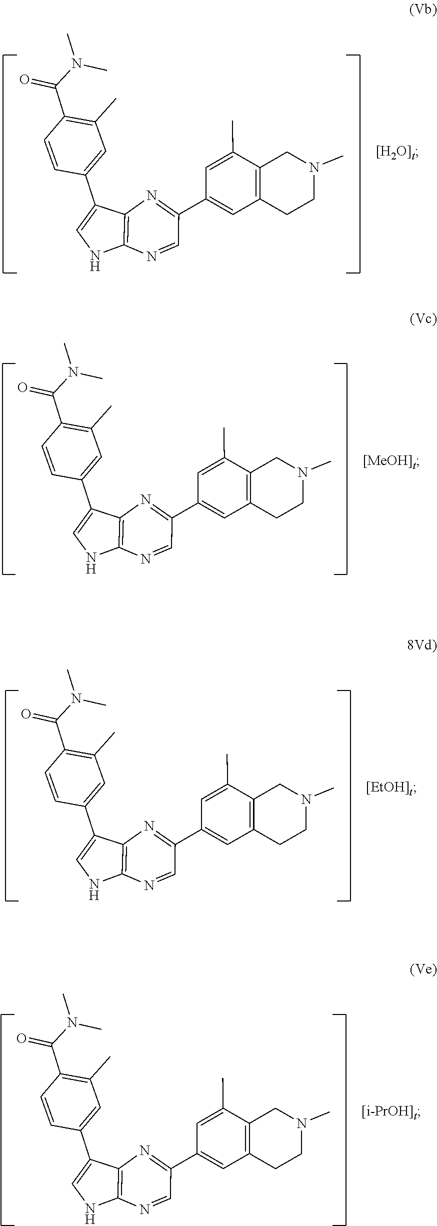

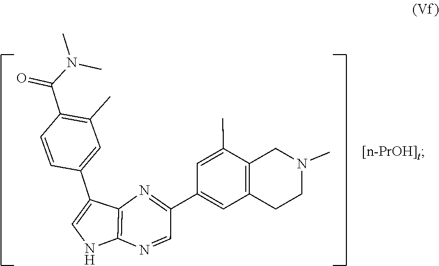

wherein t is as defined as Aspect 14 or 18;
preferably, t is about 0, 0.1, 0.14, 0.17, 0.2, 0.25, 0.3, 0.4, 0.45, 0.5, 0.50, 0.55, 0.57, 0.6, 0.65, 0.7, 0.8, 0.9, 0.95, 0.98, 1.02, 1.05, 1.08, 1.09, 1.0, 1.1, 1.11, 1.12, 1.2, 1.3, 1.4, 1.45, 1.5, 1.55, 1.6, 1.7, 1.8, 1.9, 2.0, 2.1, 2.2, 2.25, 2.3, 2.31, 2.4, 2.5, 2.6, 2.7, 2.8, 2.9, or 3.0.

Aspect 20. The crystalline form of Aspect 19, which is selected from
Freeform Type A, characterized by a powder X-ray diffraction pattern comprising three, four, five, six, seven, eight, nine or more diffraction peaks having 2θ angle values independently selected from the group consisting of 9.10±0.2, 9.84±0.2, 10.61±0.2, 11.28±0.2, 12.87±0.2, 13.60±0.2, 14.65±0.2, 15.26±0.2, 15.93±0.2, 17.74±0.2, 18.41±0.2, 18.67±0.2, 19.12±0.2, 19.92±0.2, 21.30±0.2, 21.79±0.2, 22.85±0.2, 24.47±0.2, 26.01±0.2, 26.86±0.2 and 37.67±0.2 degrees; or
Freeform Type E, characterized by a powder X-ray diffraction pattern comprising three, four, five, six, seven, eight, nine or more diffraction peaks having 2θ angle values independently selected from the group consisting of 6.64±0.2, 8.65±0.2, 10.06±0.2, 10.94±0.2, 12.86±0.2, 13.84±0.2, 15.75±0.2, 16.40±0.2, 17.30±0.2, 18.00±0.2, 19.88±0.2, 20.44±0.2, 21.25±0.2, 21.94±0.2, 23.02±0.2, 23.38±0.2, 23.99±0.2, 25.14±0.2, 26.09±0.2, 26.73±0.2, 26.98±0.2 and 28.43±0.2 degrees; or
Freeform Type F, characterized by a powder X-ray diffraction pattern comprising three, four, five, six, seven, eight, nine or more diffraction peaks having 2θ angle values independently selected from the group consisting of 6.65±0.2, 7.14±0.2, 8.45±0.2, 10.88±0.2, 13.29±0.2, 13.46±0.2, 13.92±0.2, 14.46±0.2, 16.63±0.2, 16.96±0.2, 17.23±0.2, 17.57±0.2, 18.06±0.2, 18.98±0.2, 19.48±0.2, 19.66±0.2, 20.84±0.2, 21.66±0.2, 22.98±0.2, 23.33±0.2, 24.09±0.2, 24.35±0.2, 24.85±0.2, 25.53±0.2, 26.09±0.2, 26.72±0.2, 27.81±0.2, 28.31±0.2 and 28.79±0.2 degrees; or
Freeform Type I, characterized by a powder X-ray diffraction pattern comprising three, four, five, six, seven, eight, nine or more diffraction peaks having 2θ angle values independently selected from the group consisting of 5.84±0.2, 7.61±0.2, 9.11±0.2, 11.66±0.2, 13.61±0.2, 14.04±0.2, 14.35±0.2, 15.17±0.2, 15.85±0.2, 16.56±0.2, 17.35±0.2, 17.76±0.2, 18.30±0.2, 18.73±0.2, 19.23±0.2, 20.15±0.2, 20.67±0.2, 21.01±0.2, 21.58±0.2, 22.34±0.2, 23.79±0.2, 24.16±0.2, 24.90±0.2, 25.64±0.2, 26.06±0.2, 26.80±0.2 and 27.78±0.2 degrees; or
Freeform Type N, characterized by a powder X-ray diffraction pattern comprising three, four, five, six, seven, eight, nine or more diffraction peaks having 2θ angle values independently selected from the group consisting of 7.18±0.2, 7.78±0.2, 9.37±0.2, 14.38±0.2, 14.80±0.2, 15.47±0.2 and 21.64±0.2 degrees; or
Freeform Type W, characterized by a powder X-ray diffraction pattern comprising three, four, five, six, seven, eight, nine or more diffraction peaks having 2θ angle values independently selected from the group consisting of 6.62±0.2, 8.46±0.2, 11.11±0.2, 12.20±0.2, 13.24±0.2, 13.75±0.2, 14.26±0.2, 15.17±0.2, 15.42±0.2, 15.99±0.2, 16.43±0.2, 17.05±0.2, 17.63±0.2, 17.83±0.2, 19.30±0.2, 19.76±0.2, 20.10±0.2, 21.12±0.2, 22.40±0.2, 23.22±0.2, 23.78±0.2, 24.10±0.2, 24.39±0.2, 25.25±0.2, 25.89±0.2, 27.04±0.2, 27.37±0.2, 28.29±0.2, 28.80±0.2 and 29.41±0.2 degrees; or
Freeform Type Z, characterized by a powder X-ray diffraction pattern comprising three, four, five, six, seven, eight, nine or more diffraction peaks having 2θ angle values independently selected from the group consisting of 7.20±0.2, 10.06±0.2, 11.59±0.2, 13.72±0.2, 14.49±0.2, 15.85±0.2, 16.06±0.2, 17.38±0.2, 18.04±0.2, 19.60±0.2, 20.76±0.2, 21.56±0.2, 22.98±0.2, 23.49±0.2, 24.51±0.2, and 28.40±0.2 degrees.

Aspect 21. The crystalline form of Aspect 19, which is selected from
Freeform Type A, characterized by a powder X-ray diffraction pattern comprising diffraction peaks having 2θ angle values of 5.33±0.2, 10.61±0.2, and 12.87±0.2 degrees; preferably having 20 angle values of 5.33±0.2, 10.61±0.2, 12.87±0.2, 15.93±0.2, and 26.01±0.2 degrees; more preferably having 2θ angle values of 5.33±0.2, 10.61±0.2, 12.87±0.2, 15.93±0.2, 19.12±0.2, 21.79±0.2, and 26.01±0.2 degrees; even more preferably having 2θ angle values of 5.33±0.2, 10.61±0.2, 12.87±0.2, 15.93±0.2, 18.41±0.2, 19.12±0.2, 21.30±0.2, 21.79±0.2, and 26.01±0.2 degrees; even more preferably having 2θ angle values of 5.33±0.2, 10.61±0.2, 12.87±0.2, 15.26±0.2, 15.93±0.2, 18.41±0.2, 18.67±0.2, 19.12±0.2, 21.30±0.2, 21.79±0.2, and 26.01±0.2 degrees; or
Freeform Type F, characterized by a powder X-ray diffraction pattern comprising diffraction peaks having 2θ angle values of 7.14±0.2, 8.45±0.2, and 24.35±0.2 degrees; preferably having 2θ angle values of 7.14±0.2, 8.45±0.2, 13.29±0.2, 16.63±0.2, and 24.35±0.2 degrees; more preferably having 2θ angle values of 7.14±0.2, 8.45±0.2, 13.29±0.2, 16.63±0.2, 16.96±0.2, 22.98±0.2, and 24.35±0.2 degrees; even more preferably having 2θ angle values of 7.14±0.2, 8.45±0.2, 13.29±0.2, 13.46±0.2, 13.92±0.2, 16.63±0.2, 16.96±0.2, 22.98±0.2, and 24.35±0.2 degrees; even more preferably having 2θ angle values of 6.65±0.2, 7.14±0.2, 8.45±0.2, 13.29±0.2, 13.46±0.2, 13.92±0.2, 16.63±0.2, 16.96±0.2, 22.98±0.2, 24.35±0.2, and 26.09±0.2 degrees; or
Freeform Type I, characterized by a powder X-ray diffraction pattern comprising diffraction peaks having 2θ angle values of 5.84±0.2, 16.56±0.2, and 25.64±0.2 degrees; preferably having 2θ angle values of 5.84±0.2, 14.04±0.2, 14.35±0.2, 16.56±0.2, and 25.64±0.2 degrees; more preferably having 20 angle values of 5.84±0.2, 14.04±0.2, 14.35±0.2, 15.85±0.2, 16.56±0.2, 25.64±0.2, and 26.06±0.2 degrees; even more preferably having 2θ angle values of 5.84±0.2, 7.61±0.2, 11.66±0.2, 14.04±0.2, 14.35±0.2, 15.85±0.2, 16.56±0.2, 25.64±0.2, and 26.06±0.2 degrees; even more preferably having 20 angle values of 5.84±0.2, 16.56±0.2, 25.64±0.2, 14.04±0.2, 14.35±0.2, 26.06±0.2, 15.85±0.2, 7.61±0.2, 11.66±0.2, 17.76±0.2, and 15.17±0.2 degrees.

Aspect 22. A crystalline form of Aspects 14, wherein the crystalline form is Formula VIa

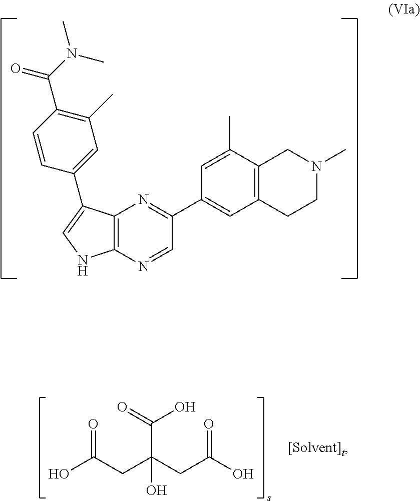

wherein [solvent], s and t are as defined as Aspect 14;
preferably, the crystalline form is Formula VIb, VIc, VId, VIe, VIf or VIg,

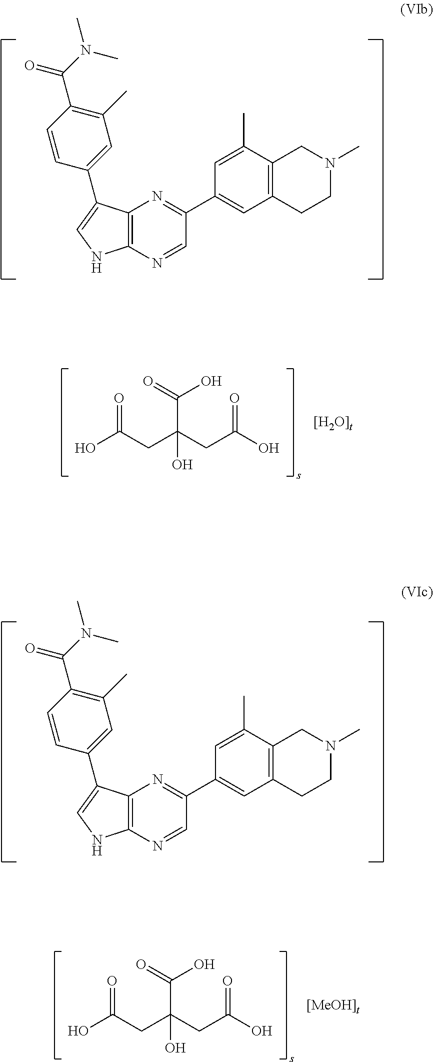

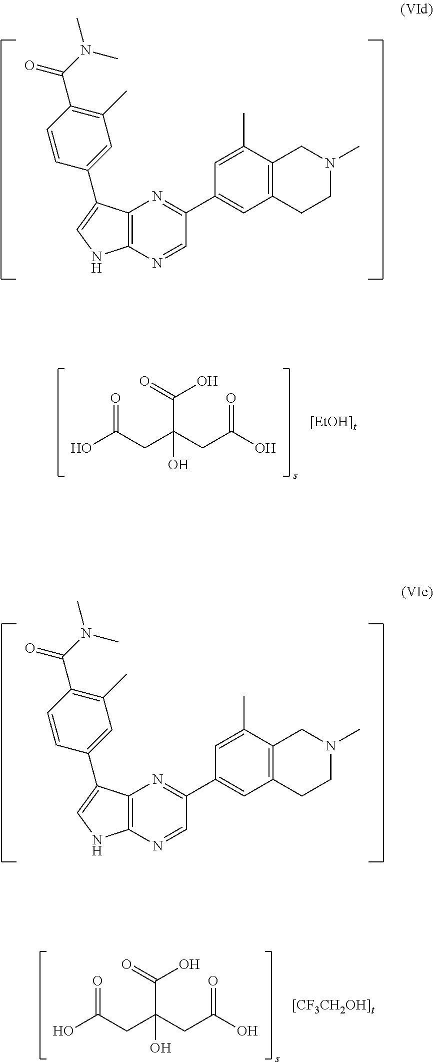

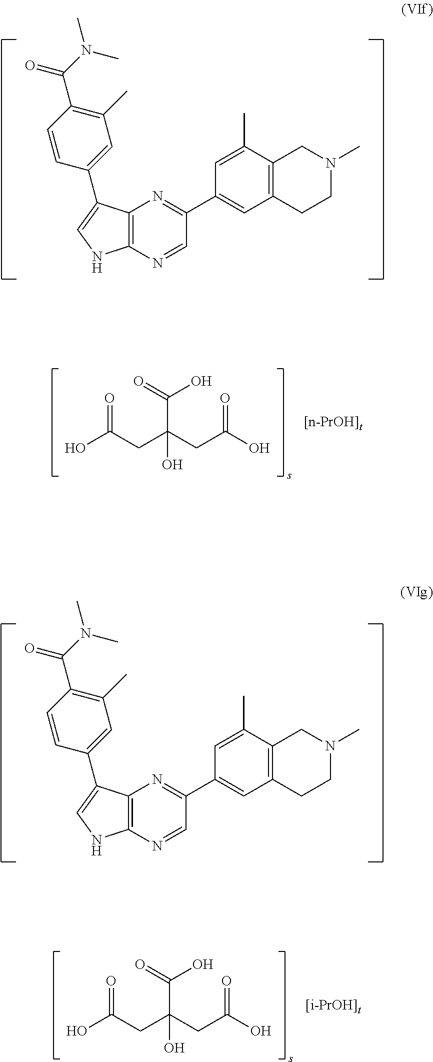

wherein s is about 0.3, 0.33, 0.40, 0.44, 0.45, 0.50, 0.55, 0.56, 0.60, 0.63, 0.65, 0.66, 0.67, 0.70, 0.75, 0.76, 0.80, 0.85, 0.90, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.20, 1.3, 1.4, 1.45, 1.5, 1.55, 1.67, 1.8, 1.90, 1.95, or 2.0; t is about 0, 0.1, 0.14, 0.17, 0.2, 0.25, 0.3, 0.4, 0.45, 0.5, 0.50, 0.55, 0.57, 0.6, 0.65, 0.7, 0.8, 0.9, 0.95, 0.98, 1.02, 1.05, 1.08, 1.09, 1.0, 1.1, 1.11, 1.12, 1.2, 1.3, 1.4, 1.45, 1.5, 1.55, 1.6, 1.7, 1.8, 1.9, 2.0, 2.1, 2.2, 2.25, 2.3, 2.31, 2.4, 2.5, 2.6, 2.7, 2.8, 2.9, or 3.0.

Aspect 23. The crystalline form of Aspect 22, which is selected from
citrate salt Type A, characterized by a powder X-ray diffraction pattern comprising three, four, five, six, seven, eight, nine or more diffraction peaks having 2θ angle values independently selected from the group consisting of 4.95±0.2, 6.59±0.2, 9.03±0.2, 9.98±0.2, 10.84±0.2, 13.02±0.2, 13.23±0.2, 14.56±0.2, 14.96±0.2, 15.55±0.2, 15.94±0.2, 16.71±0.2, 17.56±0.2, 19.22±0.2, 20.09±0.2, 20.60±0.2, 21.59±0.2, 21.96±0.2, 22.33±0.2, 22.52±0.2, 23.26±0.2, 24.17±0.2, 24.41±0.2, 25.11±0.2, 26.08±0.2, 26.88±0.2, 27.43±0.2, 27.92±0.2, 30.09±0.2 and 30.75±0.2 degrees; or
citrate salt Type B, characterized by a powder X-ray diffraction pattern comprising three, four, five, six, seven, eight, nine or more diffraction peaks having 2θ angle values independently selected from the group consisting of 6.58±0.2, 8.09±0.2, 9.94±0.2, 10.91±0.2, 13.42±0.2, 14.24±0.2, 14.82±0.2, 16.21±0.2, 18.09±0.2, 18.69±0.2, 19.86±0.2, 20.30±0.2, 20.60±0.2, 21.50±0.2, 22.20±0.2 and 22.99±0.2 degrees; or
citrate salt Type C, characterized by a powder X-ray diffraction pattern comprising three, four, five, six, seven, eight, nine or more diffraction peaks having 2θ angle values independently selected from the group consisting of 4.26±0.2, 5.40±0.2, 6.02±0.2, 6.61±0.2, 8.47±0.2, 10.80±0.2, 12.05±0.2, 12.70±0.2, 15.52±0.2, 16.96±0.2, 19.55±0.2 and 21.24±0.2 degrees; or
citrate salt Type E, characterized by a powder X-ray diffraction pattern comprising three, four, five, six, seven, eight, nine or more diffraction peaks having 2θ angle values independently selected from the group consisting of 6.75±0.2, 8.67±0.2, 9.16±0.2, 13.49±0.2, 13.73±0.2, 14.95±0.2, 16.09±0.2, 17.54±0.2, 18.42±0.2, 18.61±0.2, 20.25±0.2, 20.64±0.2, 21.66±0.2, 22.61±0.2, 22.92±0.2, 23.58±0.2, 23.92±0.2, 25.06±0.2, 25.59±0.2, 26.11±0.2, 27.24±0.2, 28.82±0.2, 29.75±0.2, 32.31±0.2 and 33.75±0.2 degrees; or
citrate salt Type F, characterized by a powder X-ray diffraction pattern comprising three, four, five, six, seven, eight, nine or more diffraction peaks having 2θ angle values independently selected from the group consisting of 4.95±0.2, 7.24±0.2, 9.14±0.2, 9.82±0.2, 13.42±0.2, 14.05±0.2, 14.57±0.2, 14.89±0.2, 15.63±0.2, 17.10±0.2, 20.19±0.2, 21.08±0.2 and 21.92±0.2 degrees.

Aspect 24. The crystalline form of Aspect 22, which is selected from
citrate salt Type A, characterized by a powder X-ray diffraction pattern comprising diffraction peaks having 2θ angle values of 14.96±0.2, 17.56±0.2 and 26.08±0.2 degrees; preferably having 20 angle values of 14.96±0.2, 17.56±0.2, 22.33±0.2, 22.52±0.2 and 26.08±0.2 degrees; more preferably having 2θ angle values of 9.03±0.2, 14.96±0.2, 15.55±0.2, 17.56±0.2, 22.33±0.2, 22.52±0.2 and 26.08±0.2 degrees; even more preferably having 2θ angle values of 9.03±0.2, 10.84±0.2, 14.96±0.2, 15.55±0.2, 17.56±0.2, 20.60±0.2, 22.33±0.2, 22.52±0.2 and 26.08±0.2 degrees; even more preferably having 2θ angle values of 9.03±0.2, 9.98±0.2, 10.84±0.2, 14.96±0.2, 15.55±0.2, 17.56±0.2, 20.60±0.2, 22.33±0.2, 22.52±0.2, 23.26±0.2 and 26.08±0.2 degrees; or
citrate salt Type B, characterized by a powder X-ray diffraction pattern comprising diffraction peaks having 2θ angle values of 6.58±0.2, 13.42±0.2, and 14.82±0.2 degrees; preferably having 20 angle values of 6.58±0.2, 13.42±0.2, 14.24±0.2, 14.82±0.2, and 19.86±0.2 degrees; more preferably having 2θ angle values of 6.58±0.2, 13.42±0.2, 14.24±0.2, 14.82±0.2, 16.21±0.2, 19.86±0.2, and 20.30±0.2 degrees; even more preferably having 2θ angle values of 6.58±0.2, 8.09±0.2, 13.42±0.2, 14.24±0.2, 14.82±0.2, 16.21±0.2, 19.86±0.2, 20.30±0.2, and 20.60±0.2; degrees even more preferably having 2θ angle values of 6.58±0.2, 8.09±0.2, 9.94±0.2, 13.42±0.2, 14.24±0.2, 14.82±0.2, 16.21±0.2, 18.09±0.2, 19.86±0.2, 20.30±0.2, and 20.60±0.2 degrees; or
citrate salt Type C, characterized by a powder X-ray diffraction pattern comprising diffraction peaks having 2θ angle values of 4.26±0.2, 8.47±0.2, and 12.70±0.2 degrees; preferably having 2θ angle values of 4.26±0.2, 5.40±0.2, 8.47±0.2, 12.70±0.2, and 21.24±0.2 degrees; more preferably having 20 angle values of 4.26±0.2, 5.40±0.2, 8.47±0.2, 10.80±0.2, 12.70±0.2, 21.24±0.2, and 19.55±0.2 degrees; even more preferably having 2θ angle values of 4.26±0.2, 5.40±0.2, 8.47±0.2, 10.80±0.2, 12.70±0.2, 15.52±0.2, 16.96±0.2, 19.55±0.2, and 21.24±0.2 degrees; even more preferably having 2θ angle values of 4.26±0.2, 5.40±0.2, 6.02±0.2, 8.47±0.2, 10.80±0.2, 12.70±0.2, 15.52±0.2, 16.96±0.2, 19.55±0.2, 19.91±0.2, and 21.24±0.2 degrees; or
citrate salt Type E, characterized by a powder X-ray diffraction pattern comprising diffraction peaks having 2θ angle values of 9.16±0.2, 13.49±0.2, and 13.73±0.2 degrees; preferably having 20 angle values of 9.16±0.2, 13.49±0.2, 13.73±0.2, 14.95±0.2, and 22.92±0.2 degrees; more preferably having 2θ angle values of 9.16±0.2, 13.49±0.2, 13.73±0.2, 14.95±0.2, 18.42±0.2, 22.92±0.2, and 27.24±0.2 degrees; even more preferably having 2θ angle values of 8.67±0.2, 9.16±0.2, 13.49±0.2, 13.73±0.2, 14.95±0.2, 18.42±0.2, 22.61±0.2, 22.92±0.2, and 27.24±0.2 degrees; even more preferably having 2θ angle values of 8.67±0.2, 9.16±0.2, 13.49±0.2, 13.73±0.2, 14.95±0.2, 18.42±0.2, 21.66±0.2, 22.61±0.2, 22.92±0.2, 25.06±0.2, and 27.24±0.2 degrees; or
citrate salt Type F, characterized by a powder X-ray diffraction pattern comprising diffraction peaks having 2θ angle values of 13.42±0.2, 14.05±0.2, and 20.19±0.2 degrees; preferably having 20 angle values of 7.24±0.2, 13.42±0.2, 14.05±0.2, 20.19±0.2, and 21.08±0.2 degrees; more preferably having 2θ angle values of 7.24±0.2, 13.42±0.2, 14.05±0.2, 14.57±0.2, 17.10±0.2, 20.19±0.2, and 21.08±0.2 degrees; even more preferably having 2θ angle values of 7.24±0.2, 9.82±0.2, 13.42±0.2, 14.05±0.2, 14.57±0.2, 14.89±0.2, 17.10±0.2, 20.19±0.2 and 21.08±0.2 degrees; even more preferably having 2θ angle values of 4.95±0.2, 7.24±0.2, 9.82±0.2, 13.42±0.2, 14.05±0.2, 14.57±0.2, 14.89±0.2, 17.10±0.2, 20.19±0.2, 21.08±0.2 and 21.92±0.2 degrees.

Aspect 25. The crystalline form of Aspect 14, which is selected from
L-malate salt Type A, characterized by a powder X-ray diffraction pattern comprising three, four, five, six, seven, eight, nine or more diffraction peaks having 2θ angle values independently selected from the group consisting of 5.06±0.2, ±0.2, 6.97±0.2, 7.28±0.2, 10.09±0.2, 10.49±0.2, 13.36±0.2, 14.67±0.2, 15.13±0.2, 16.08±0.2, 17.02±0.2, 18.10±0.2, 18.44±0.2, 18.74±0.2, 19.54±0.2, 20.05±0.2, 20.41±0.2, 21.07±0.2, 22.35±0.2, 22.82±0.2, 23.45±0.2, 23.83±0.2, 25.36±0.2, 25.72±0.2, 28.14±0.2, 29.55±0.2, 30.57±0.2, 31.22±0.2, 32.36±0.2 and 33.38±0.2 degrees; or
succinate salt Type B, characterized by a powder X-ray diffraction pattern comprising three, four, five, six, seven, eight, nine or more diffraction peaks having 2θ angle values independently selected from the group consisting of 6.79±0.2, 9.13±0.2, 9.40±0.2, 10.22±0.2, 10.81±0.2, 12.09±0.2, 13.53±0.2, 14.25±0.2, 14.86±0.2, 15.25±0.2, 15.90±0.2, 16.55±0.2, 16.81±0.2, 17.66±0.2, 18.15±0.2, 19.06±0.2, 19.74±0.2, 20.03±0.2, 20.42±0.2, 20.71±0.2, 21.03±0.2, 22.34±0.2, 22.85±0.2, 23.22±0.2, 23.87±0.2, 24.42±0.2, 24.87±0.2, 25.19±0.2, 26.38±0.2, 26.90±0.2, 27.78±0.2, 28.13±0.2, 28.84±0.2 and 29.93±0.2 degrees; or
fumarate Type B, characterized by a powder X-ray diffraction pattern comprising three, four, five, six, seven, eight, nine or more diffraction peaks having 2θ angle values independently selected from the group consisting of 4.83±0.2, 6.66±0.2, 8.44±0.2, 9.22±0.2, 10.69±0.2, 11.60±0.2, 12.06±0.2, 12.80±0.2, 13.44±0.2, 13.86±0.2, 14.29±0.2, 15.44±0.2, 16.02±0.2, 16.33±0.2, 16.95±0.2, 17.54±0.2, 18.18±0.2, 18.46±0.2, 18.97±0.2, 19.75±0.2, 20.03±0.2, 20.57±0.2, 21.14±0.2, 21.48±0.2, 22.30±0.2, 23.15±0.2, 23.96±0.2, 24.67±0.2, 24.98±0.2, 26.91±0.2, 27.85±0.2, 28.46±0.2, 31.01±0.2, 31.45±0.2, and 35.59±0.2 degrees; or
fumarate Type D, characterized by a powder X-ray diffraction pattern comprising three, four, five, six, seven, eight, nine or more diffraction peaks having 2θ angle values independently selected from the group consisting of 3.29±0.2, 4.87±0.2, 6.71±0.2, 7.47±0.2, 8.08±0.2, 8.68±0.2, 10.08±0.2, 10.35±0.2, 12.67±0.2, 13.48±0.2, 14.01±0.2, 14.30±0.2, 14.83±0.2, 15.28±0.2, 15.55±0.2, 16.67±0.2, 17.31±0.2, 18.66±0.2, 18.95±0.2, 19.94±0.2, 20.05±0.2, 20.40±0.2, 21.28±0.2, 22.00±0.2, 23.10±0.2, 23.34±0.2, 24.18±0.2, 25.13±0.2, 25.83±0.2, 26.86±0.2, 30.52±0.2, 35.12±0.2 and 35.47±0.2 degrees; or maleate Type A, characterized by a powder X-ray diffraction pattern comprising three, four, five, six, seven, eight, nine or more diffraction peaks having 2θ angle values independently selected from the group consisting of 5.07±0.2, 8.06±0.2, 8.81±0.2, 11.70±0.2, 12.71±0.2, 13.44±0.2, 14.77±0.2, 15.25±0.2, 15.51±0.2, 16.18±0.2, 16.44±0.2, 17.32±0.2, 17.56±0.2, 19.02±0.2, 19.43±0.2, 20.92±0.2, 21.38±0.2, 22.20±0.2, 22.69±0.2, 23.48±0.2, 24.03±0.2, 24.80±0.2, 25.23±0.2, 25.99±0.2, 26.91±0.2, 27.37±0.2, 27.99±0.2, 29.49±0.2, 31.39±0.2, 32.33±0.2 and 33.13±0.2 degrees; or
hydrochloride Type A, characterized by a powder X-ray diffraction pattern comprising three, four, five, six, seven, eight, nine or more diffraction peaks having 2θ angle values independently selected from the group consisting of 5.55±0.2, 8.12±0.2, 8.41±0.2, 9.29±0.2, 11.83±0.2, 12.08±0.2, 13.47±0.2, 15.42±0.2, 15.75±0.2, 16.16±0.2, 16.53±0.2, 16.94±0.2, 18.00±0.2, 18.60±0.2, 19.84±0.2, 20.24±0.2, 21.72±0.2, 22.13±0.2, 23.08±0.2, 23.55±0.2, 24.44±0.2, 26.15±0.2, 26.38±0.2, 26.91±0.2, 27.92±0.2, 28.32±0.2, 33.12±0.2, 33.27±0.2, 34.17±0.2 and 35.28±0.2 degrees; or
hydrochloride Type C, characterized by a powder X-ray diffraction pattern comprising three, four, five, six, seven, eight, nine or more diffraction peaks having 2θ angle values independently selected from the group consisting of 6.07±0.2, 6.81±0.2, 8.31±0.2, 9.80±0.2, 12.08±0.2, 12.85±0.2, 13.17±0.2, 13.55±0.2, 13.89±0.2, 15.71±0.2, 16.11±0.2, 16.68±0.2, 18.12±0.2, 18.72±0.2, 19.34±0.2, 20.31±0.2, 20.86±0.2, 22.17±0.2, 23.89±0.2, 25.07±0.2, 25.44±0.2, 26.05±0.2, 26.46±0.2, 27.12±0.2, 27.48±0.2, 28.06±0.2, 28.77±0.2, 29.13±0.2, 29.79±0.2, 30.40±0.2, 30.71±0.2, 31.97±0.2, 33.75±0.2, 35.28±0.2 and 35.74±0.2 degrees; or
sulfate Type A, characterized by a powder X-ray diffraction pattern comprising three, four, five, six, seven, eight, nine or more diffraction peaks having 2θ angle values independently selected from the group consisting of 5.19±0.2, 6.87±0.2, 7.71±0.2, 10.28±0.2, 11.14±0.2, 13.59±0.2, 14.63±0.2, 15.35±0.2, 15.71±0.2, 16.17±0.2, 18.00±0.2, 18.24±0.2, 19.20±0.2, 20.23±0.2, 20.52±0.2, 21.30±0.2, 22.00±0.2, 22.30±0.2, 22.90±0.2, 24.94±0.2, 25.79±0.2, 28.52±0.2, 29.15±0.2 and 29.55±0.2 degrees.

Aspect 26. The crystalline form of any one of Aspects 14-25, substantially characterized by a powder X-ray diffraction pattern selected from the group consisting of FIG. 1A, FIG. 2A, FIG. 3A, FIG. 4A, FIG. 5A, FIG. 7A, FIG. 8A, FIG. 9A, FIG. 10A, FIG. 11A, FIG. 12A, FIG. 13A, FIG. 14A, FIG. 15A, FIG. 16A, FIG. 17A, FIG. 18A, FIG. 19A or FIG. 20A.

Aspect 27. A pharmaceutical composition comprising a therapeutically effective amount of crystalline form according to any one of Aspects 14-26, and optionally one or more pharmaceutically acceptable carrier(s).

Aspect 28. A method for treating or preventing a disorder or a disease selected from inflammatory disorder, autoimmune disease, or a cancer, comprising administering a subject in need thereof a therapeutically effective amount of the crystalline form according to any one of Aspect 14-26, or the pharmaceutical composition of Aspect 27.

### BRIEF DESCRIPTIONS OF THE DRAWINGS

FIG. 1A XRPD pattern of Compound A free form Type A

FIG. 1B TGA/DSC curves of Compound A free form Type A

FIG. 1C 1H NMR spectrum of Compound A free form Type A

FIG. 1D DVS plot of Compound A free form Type A

FIG. 2A XRPD overlay of Compound A free form Type F

FIG. 2B TGA/DSC curves of Compound A free form Type F

FIG. 3A XRPD pattern of Compound A free form Type I

FIG. 3B TGA/DSC curves of Compound A free form Type I

FIG. 4A XRPD overlay of Compound A free form Type N

FIG. 5A VT-XRPD overlay of freeform Type N and Type Z

FIG. 6A XRPD overlay of Compound A free form Type E

FIG. 7A VT-XRPD overlay of Compound A free form Type E and Type W

FIG. 8A XRPD pattern of Compound A L-malate salt Type A

FIG. 8B DSC thermogram of Compound A L-malate salt Type A

FIG. 8C TGA thermogram of Compound A L-malate salt Type A

FIG. 8D 1H-NMR spectrum of Compound A L-malate salt Type A

FIG. 9A XRPD pattern of Compound A citrate salt Type A

FIG. 9B DSC thermogram of Compound A citrate salt Type A

FIG. 9C TGA thermogram of Compound A citrate salt Type A

FIG. 9D 1H-NMR spectrum of Compound A citrate salt Type A

FIG. 9E DVS isotherm plot of Compound A citrate salt Type A at 25° C.

FIG. 10A XRPD pattern of Compound A fumarate Type B

FIG. 10B 1H-NMR spectrum of Compound A fumarate Type B

FIG. 11A XRPD pattern of Compound A fumarate Type D

FIG. 11B DSC thermogram of Compound A fumarate Type D

FIG. 11C TGA thermogram of Compound A fumarate Type D

FIG. 11D 1H-NMR spectrum of Compound A fumarate Type D

FIG. 12A XRPD pattern of Compound A maleate Type A

FIG. 12B 1H-NMR spectrum of Compound A maleate Type A

FIG. 13A XRPD pattern of Compound A succinate Type B

FIG. 13B 1H-NMR spectrum of Compound A succinate Type A

FIG. 14A XRPD pattern of Compound A hydrochloride Type A

FIG. 14B 1H-NMR spectrum of Compound A hydrochloride Type A

FIG. 15A XRPD pattern of Compound A hydrochloride Type C

FIG. 15B DSC thermogram of Compound A hydrochloride Type C

FIG. 15C TGA thermogram of Compound A hydrochloride Type C

FIG. 15D 1H-NMR spectrum of Compound A hydrochloride Type C

FIG. 16A XRPD pattern of Compound A sulfate Type A

FIG. 16B 1H-NMR spectrum of Compound A sulfate Type A

FIG. 17A XRPD pattern of Compound A citrate type B

FIG. 17B DSC thermogram of Compound A citrate Type B

FIG. 17C TGA thermogram of Compound A citrate Type B

FIG. 17D 1H-NMR spectrum of Compound A citrate Type B

FIG. 18A XRPD pattern of Compound A citrate Type C

FIG. 18B 1H-NMR spectrum of Compound A citrate Type C

FIG. 19A XRPD pattern of Compound A citrate Type E

FIG. 19B 1H-NMR spectrum of Compound A citrate Type E

FIG. 20A XRPD pattern of Compound A citrate Type F

FIG. 20B 1H-NMR spectrum of Compound A citrate Type F

### DETAILED DESCRIPTION OF THE INVENTION

Although a freebase may theoretically form pharmaceutically acceptable salts with many acids, Compound A as a specific freebase disclosed herein has been found cannot form a salt with some acids (such as L-aspartic acid) or cannot form a crystalline salt with the desired crystallinity. Inventors surprisingly found that the citrate salt of Compound A has a good crystallinity, safety and production compatibility.

For crystalline forms described above, only the main peaks (i.e., the most characteristic, significant, unique and/or reproducible peaks) are summarized; additional peaks may be obtained from the diffraction spectra by conventional methods. The main peaks described above can be reproduced within the margin of error (+ or −2 at the last given decimal place, or + or −0.2 at the stated value).

The method for preparing the free base of Compound A is disclosed in WO2021000925 A1. For the above-mentioned crystalline forms, the crystallization step can be conducted in an appropriate solvent system containing at least one solvent by evaporation of solvent, cooling and/or by addition of anti-solvents (solvents that are less able to solubilize the Compound A or its salts, including but not limited to those described herein) to achieve super-saturation in the solvent system.

Crystallization may be done with or without seed crystals, which is described in the present invention.

In an embodiment in this aspect, provided herewith is the citrate of Compound A, preferably in the above-mentioned crystalline forms, more preferably in the crystalline forms of Types A, B, C, E and F, even more preferably in the crystalline forms of Types A, B and E, most preferably in the crystalline form of Type A.

In an embodiment in this aspect, provided herewith is the free form of Compound A, preferably in the above-mentioned crystalline forms, more preferably in the crystalline forms of Types A, E, F, I, N, W and Z, even more preferably in the crystalline forms of Types A, F and I, most preferably in the crystalline form of Type A.

The individual crystalline forms provided by the present invention develop under specific conditions dependent on the particular thermodynamic and equilibrium properties of the crystallization process. Therefore, a person skilled in the art will know that the crystals formed are a consequence of the kinetic and thermodynamic properties of the crystallization process. Under certain conditions (such as in a specific solvent), a particular crystalline form may have better properties than another crystalline form (or in fact have better properties than any other crystalline forms).

In another aspect, provided herein is a pharmaceutical composition each containing an effective amount of citrate of Compound A, preferably in any of the above-described crystalline forms. The active compound can be 1-99% (by weight), preferably 1-70% (by weight), or more preferably 1-50% (by weight), or most preferably, 5-40% (by weight), of the composition.

In another aspect, provided herein is the use of the above-described salt or crystalline forms of Compound A in the manufacture of medicaments for the treatment of a cancer associated with HPK1 inhibition.

The term “about” as used herein, unless indicated otherwise, denotes that a numer (e.g., temperature, pH, volume, etc.) can vary within +10%, preferably within +5%.

A solvate herein is defined as a compound formed by solvation, for example as a combination of solvent molecules with molecules or ions of a solute. The known solvent molecules include water, alcohols and other polar organic solvents. Alcohols inculde methanol, ethanol, n-propanol, iso-propanol, n-butanol, iso-butanol and t-butanol. The preferred solvent is typically water. The solvate compounds formed by solvation with water are sometimes termed as hydrates.

The following synthetic methods, specific examples, and efficacy tests further describe certain aspects of the present invention. They shall not limit or restrict the scope of the present invention in any way.

In some embodiments, the crystalline form has a crystalline purity at least about 80%, preferably at least about 90%, preferably at least about 95% crystalline purity, preferably about 97% crystalline purity, more preferably about 99% or more crystalline purity, and most preferably about 100% crystalline purity.

The term “crystalline purity,” as used herein, means the percentage of a particular crystalline form of a compound in a sample, which may contain the amorphous form of the compound, one or more other crystalline forms of the compound (other than the particular crystalline form of the compound), or a mixture thereof. Crystalline purity is determined by X-ray powder diffraction (XRPD), Infrared Raman spectroscopy and other solid state methods.

### EXAMPLES

The examples below are intended to be exemplary and efforts have been made to ensure accuracy with respect to numbers used (for example, amounts, temperature, etc.), but some experimental errors and deviations should be accounted for within the knowledge of a person skilled in the art. Unless indicated otherwise, temperature is in degrees Centigrade. Reagents were purchased from commercial suppliers such as Sigma-Aldrich, Alfa Aesar, or TCI, and were used without further purification unless otherwise indicated.

¹H NMR spectra were recorded on a Bruker instrument operating at noted frequency with preset pulse sequences.

Powder X-ray diffraction (XRPD) analysis was conducted using one of below methods:

PANalytical X'Pert3 diffractometer equipped with a copper radiation source was used. Sample was spread on the middle of a zero-background Si holder and rotated during collection. The divergence slit was set at 1/8° continuous illumination. The X-ray tube voltage and amperage were set to 45 kV and 40 mA respectively. Data was collected at the Cu wavelength (Kα1: 1.540598 Å; Kα2: 1.544426 Å; Kα2/Kα1 intensity ratio: 0.50) in the Theta-Theta goniometer from 3.0 to 40.0 degrees, 2-Theta using a step size of 0.0263 degrees.

PANalytical Empyrean diffractometer equipped with a copper radiation source was used. Sample was spread on the middle of a zero-background Si holder and rotated during collection. The divergence slit was set at automatic continuous illumination. The X-ray tube voltage and amperage were set to 45 kV and 40 mA respectively. Data was collected at the Cu wavelength (Kα1: 1.540598 Å; Kα2: 1.544426 Å; Kα2/Kα1 intensity ratio: 0.50) in the Theta-Theta goniometer from 3.0 to 40.0 degrees, 2-Theta using a step size of 0.0167 degrees.

Bruker D8 Advance diffractometer equipped with a copper radiation source was used. Sample was spread on the middle of a zero-background Si holder and rotated during collection. The divergence slit was set at 10.0 mm continuous illumination. The X-ray tube voltage and amperage were set to 40 kV and 40 mA respectively. Data was collected at the Cu wavelength (Kα: 1.5418 Å) in the Theta-Theta goniometer from 2.0 to 40.0 degrees, 2-Theta using a step size of 0.02 degrees.

Thermogravimetric (TGA) analysis was conducted using one of below methods:

TA TGA 5500 thermogravimetric analyzer was used. Sample was weighted to an open aluminum pan and protected with nitrogen flow. The sample was heated from ambient temperature to 350° C. with 10° C./min heating rate.

TA TGA 5500 thermogravimetric analyzer was used. Sample was weighted to an open aluminum pan and protected with nitrogen flow. The sample was heated from ambient temperature to 300° C. with 10° C./min heating rate.

Differential scanning calorimetry (DSC) analysis was conducted using TA DSC 2500 differential scanning calorimeter. Sample was weighted to a crimped aluminum pan and protected with nitrogen flow. The sample was heated from ambient temperature to target temperature with 10° C./min heating rate.

Dynamic vapor sorption (DVS) analysis was conducted using SMS DVS Intrinsic using one of below methods:

Sample was weighted to the microbalance. The relative humidity was set to 0% with N₂ flow of 200 sccm. The equilibrium was assumed when the weight change of the sample was <0.001 wt % in 10 min or by a maximum equilibration time of 180 minutes. The relative humidity was then progressively increased to 90% in 10% RH increments, then to 95%, then to 90%, then followed by a decrease to a final RH of 0% in 10% RH decrements. Between two equilibrium steps, dm/dt is 0.002%/min. At each equilibrium step, the equilibrium was assumed when the weight change of the sample was <0.001 wt % in 10 min or by a maximum equilibration time of 180 minutes.

Sample was weighted to the microbalance. The relative humidity was set to the closes decile of ambient humidity with N₂ flow of 200 sccm. The equilibrium was assumed when the weight change of the sample was <0.001 wt % in 10 min or by a maximum equilibration time of 180 minutes. The relative humidity was then progressively increased to 90% in 10% RH increments, then to 95%, then to 90%, then to 0% in 10% RH decrements, then to 90% in 10% RH increments, then followed by a increase to a final RH of 95%. Between two equilibrium steps, dm/dt is 0.002%/min. At each equilibrium step, the equilibrium was assumed when the weight change of the sample was <0.001 wt % in 10 min or by a maximum equilibration time of 180 minutes.

Sample was weighted to the microbalance. The relative humidity was set to 40% with N₂ flow of 200 sccm. The equilibrium was assumed when the weight change of the sample was <0.001 wt % in 60 min or by a maximum equilibration time of 360 minutes. The relative humidity was then progressively decreased to 0% in 10% RH decrements, then to 90% in 10% RH increments, then to 95%, then to 90%, then followed by a decrease to a final RH of 40% in 10% RH decrements. Between two equilibrium steps, dm/dt is 0.002%/min. At each equilibrium step, the equilibrium was assumed when the weight change of the sample was <0.001 wt % in 60 min or by a maximum equilibration time of 360 minutes.

In the following examples, the abbreviations below may be used:

| | |
|---|---|
| AcOH | Acetic acid |
| ACN or MeCN | Acetonitrile |
| Aq | Aqueous |
| DMF | N,N-Dimethylformamide |
| DMSO | Dimethyl sulfoxide |
| EtOAc | Ethyl acetate |
| EtOH | Ethanol |
| Et₂O or ether | Diethyl ether |
| g | grams |
| h or hr | hour |
| HCl | Hydrochloric acid |
| HPLC | High-performance liquid chromatography |
| IPA | 2-propanol |
| i-PrOH | Isopropyl alcohol |
| mg | milligrams |
| mL | milliliters |
| mmol | millimole |
| MeOH | Methanol |
| Min | minutes |
| PE | petroleum ether |
| RH | Relative Humidity |
| Rt | Retention time |
| Rt or rt | Room temperature |
| TFA | Trifluoroacetic acid |
| THF | tetrahydrofuran |
| TLC | thin layer chromatography |
| μL | microliters |

### Example 1: Preparation and XRPD Data of Compound a Free Form Type a

Compound A (110 g, obtained through the same way published in WO2021000925 A1) was dissolved in refluxing absolute EtOH (2000 mL) until all solid dissolved. The solution was cooled down to 10° C. and stirred for 5 h, by when a large quantity of solid appeared. The solid was collected by filtration and the material was recrystallized again with absolute EtOH (2200 mL). The solid was collected by filtration, then the solid was dried under vacuum to give Compound A as free form type A (62 g 54%0).

TABLE 1
Characteristic XRPD information of Compound A free form type A

| Angle [°2θ] | d-spacing [Å] | Relative Intensity [%] |
|---|---|---|
| 5.33 | 16.57 | 100.00 |
| 9.10 | 9.72 | 0.72 |
| 9.84 | 8.99 | 0.54 |
| 10.61 | 8.34 | 7.53 |
| 11.28 | 7.84 | 1.49 |
| 12.87 | 6.88 | 8.96 |
| 13.60 | 6.51 | 1.07 |
| 14.65 | 6.05 | 1.68 |
| 15.26 | 5.81 | 2.23 |
| 15.93 | 5.56 | 6.33 |
| 17.74 | 5.00 | 1.78 |
| 18.41 | 4.82 | 2.25 |
| 18.67 | 4.75 | 2.22 |
| 19.12 | 4.64 | 2.71 |
| 19.92 | 4.46 | 0.98 |
| 21.30 | 4.17 | 2.51 |
| 21.79 | 4.08 | 2.63 |
| 22.85 | 3.89 | 1.79 |
| 24.47 | 3.64 | 1.91 |
| 26.01 | 3.43 | 3.96 |
| 26.86 | 3.32 | 2.07 |
| 37.67 | 2.39 | 0.86 |

Solid form stability of freeform Type A was investigated in process solvents EtOH/H₂O and MeOH/H₂O. No form change of Type A was observed in MeOH at 50° C. No form change was observed under the laboratory conditions, which indicated the good physical stability of the sample. As for HPLC area purity, ˜1.2 area % decrease was observed in Vis (10000 Lux) for 5 days. Purity change in all the rest conditions (40° C./75% RH/open for 2 weeks, 60° C./sealed for 10 days, RT/92.5% RH/open for 10 days, 25° C./60% RH/open for 8 weeks and UV (290 wμ/cm2) for 3 days) was less than 0.4 area %.

### Example 2: Preparation and XRPD Data of Compound a Free Form Type F

Compound A free form type A (19.6 mg) was suspended in acetonitrile (0.50 mL) in a glass vial. The slurry was stirred at 50° C. using magnetic stirring for 1 week. The resultant solid was separated to give Compound A as free form type F.

TABLE 2
Characteristic XRPD information of Compound A free form type F

| Angle [°2θ] | d-spacing [Å] | Relative Intensity [%] |
|---|---|---|
| 6.65 | 13.30 | 10.62 |
| 7.14 | 12.39 | 32.68 |
| 8.45 | 10.47 | 100.00 |
| 10.88 | 8.13 | 0.73 |
| 13.29 | 6.66 | 27.89 |
| 13.46 | 6.58 | 12.48 |
| 13.92 | 6.36 | 20.88 |
| 14.46 | 6.13 | 3.27 |
| 16.63 | 5.33 | 28.66 |
| 16.96 | 5.23 | 21.01 |
| 17.23 | 5.15 | 2.27 |
| 17.57 | 5.05 | 6.16 |
| 18.06 | 4.91 | 6.84 |
| 18.98 | 4.68 | 2.81 |
| 19.48 | 4.56 | 3.51 |
| 19.66 | 4.52 | 4.88 |
| 20.84 | 4.26 | 2.31 |
| 21.66 | 4.10 | 6.66 |
| 22.98 | 3.87 | 22.35 |
| 23.33 | 3.81 | 1.92 |
| 24.09 | 3.69 | 7.69 |
| 24.35 | 3.66 | 31.53 |
| 24.85 | 3.58 | 1.89 |
| 25.53 | 3.49 | 1.93 |
| 26.09 | 3.42 | 10.85 |
| 26.72 | 3.34 | 1.85 |
| 27.81 | 3.21 | 2.25 |
| 28.31 | 3.15 | 4.04 |
| 28.79 | 3.10 | 2.05 |

### Example 3: Preparation and XRPD Data of Compound a Free Form Type I

Compound A free form type A (20.2 mg) was suspended in toluene (0.50 mL) in a glass vial. The slurry was stirred at 50° C. using magnetic stirring for 1 week. The resultant solid was separated to give Compound A as free form type I.

TABLE 3
Characteristic XRPD information of Compound A free form type I

| Angle [°2θ] | d-spacing [Å] | Relative Intensity [%] |
|---|---|---|
| 5.84 | 15.14 | 100.00 |
| 7.61 | 11.62 | 29.18 |
| 9.11 | 9.71 | 1.38 |
| 11.66 | 7.59 | 27.35 |
| 13.61 | 6.51 | 9.48 |
| 14.04 | 6.31 | 60.05 |
| 14.35 | 6.17 | 38.10 |
| 15.17 | 5.84 | 15.30 |
| 15.85 | 5.59 | 29.69 |
| 16.56 | 5.35 | 70.17 |
| 17.35 | 5.11 | 7.77 |
| 17.76 | 4.99 | 16.27 |
| 18.30 | 4.85 | 7.97 |
| 18.73 | 4.74 | 3.03 |
| 19.23 | 4.62 | 9.35 |
| 20.15 | 4.41 | 4.49 |
| 20.67 | 4.30 | 4.93 |
| 21.01 | 4.23 | 7.85 |
| 21.58 | 4.12 | 10.65 |
| 22.34 | 3.98 | 11.57 |
| 23.79 | 3.74 | 2.40 |
| 24.16 | 3.68 | 8.00 |
| 24.90 | 3.58 | 3.81 |
| 25.64 | 3.47 | 64.27 |
| 26.06 | 3.42 | 36.79 |
| 26.80 | 3.33 | 3.30 |
| 27.78 | 3.21 | 7.25 |

### Example 4: Preparation and XRPD Data of Compound a Free Form Type N

Compound A free form type A (20.9 mg) was dissolved in EtOH/water (1.0 mL, 4:1) in a vial with a pierced cap for slow evaporation. After 1 week, solid was separated to give Compound A as free form type N.

TABLE 4
Characteristic XRPD information of Compound A free form type N

| Angle [°2θ] | d-spacing [Å] | Relative Intensity [%] |
|---|---|---|
| 7.18 | 12.32 | 100.00 |
| 7.78 | 11.36 | 54.77 |
| 9.37 | 9.44 | 18.47 |
| 14.38 | 6.16 | 10.54 |
| 14.80 | 5.99 | 9.83 |
| 15.47 | 5.73 | 24.65 |
| 21.64 | 4.11 | 4.09 |

### Example 5: Preparation and XRPD Data of Compound a Free Form Type Z

Compound A free form type N was heated to 100° C. under nitrogen to give Compound A as free form type Z.

TABLE 5
Characteristic XRPD information of Compound A free form type Z

| Angle [°2θ] | d-spacing [Å] | Relative Intensity [%] |
|---|---|---|
| 7.20 | 12.28 | 93.89 |
| 10.06 | 8.79 | 71.52 |
| 11.59 | 7.63 | 13.83 |
| 13.72 | 6.45 | 60.80 |
| 14.49 | 6.11 | 25.21 |
| 15.85 | 5.59 | 86.54 |
| 16.06 | 5.52 | 100.00 |
| 17.38 | 5.10 | 13.38 |
| 18.04 | 4.92 | 21.04 |
| 19.60 | 4.53 | 19.34 |
| 20.76 | 4.28 | 13.56 |
| 21.56 | 4.12 | 14.71 |
| 22.98 | 3.87 | 18.76 |
| 23.49 | 3.79 | 49.16 |
| 24.51 | 3.63 | 26.51 |
| 28.40 | 3.14 | 9.36 |

### Example 6: Preparation and XRPD Data of Compound a Free Form Type E

Compound A free form type A (20.2 mg) was suspended in acetone (0.50 mL) in a glass vial. The slurry was stirred at 50° C. using magnetic stirring for 1 week. The resultant solid was separated to give Compound A as free form type E.

TABLE 6
Characteristic XRPD information of Compound A free form type E

| Angle [°2θ] | d-spacing [Å] | Relative Intensity [%] |
|---|---|---|
| 6.64 | 13.31 | 100.00 |
| 8.65 | 10.22 | 97.78 |
| 10.06 | 8.80 | 25.48 |
| 10.94 | 8.09 | 7.44 |
| 12.86 | 6.89 | 2.55 |
| 13.84 | 6.40 | 17.08 |
| 15.75 | 5.63 | 16.33 |
| 16.40 | 5.40 | 23.05 |
| 17.30 | 5.13 | 10.84 |
| 18.00 | 4.93 | 14.63 |
| 19.88 | 4.47 | 37.28 |
| 20.44 | 4.34 | 10.45 |
| 21.25 | 4.18 | 40.03 |
| 21.94 | 4.05 | 7.94 |
| 23.02 | 3.86 | 12.31 |
| 23.38 | 3.80 | 24.44 |
| 23.99 | 3.71 | 14.81 |
| 25.14 | 3.54 | 23.65 |
| 26.09 | 3.42 | 6.82 |
| 26.73 | 3.34 | 10.83 |
| 26.98 | 3.30 | 19.37 |
| 28.43 | 3.14 | 9.29 |

### Example 7: Preparation and XRPD Data of Compound a Free Form Type W

Compound A free form type E was heated to 100° C. under nitrogen to give Compound A as free form type W.

TABLE 7
Characteristic XRPD information of Compound A free form type W

| Angle [°2θ] | d-spacing [Å] | Relative Intensity [%] |
|---|---|---|
| 6.62 | 13.36 | 100.00 |
| 8.46 | 10.45 | 99.81 |
| 11.11 | 7.97 | 2.48 |
| 12.20 | 7.25 | 4.55 |
| 13.24 | 6.69 | 30.20 |
| 13.75 | 6.44 | 9.71 |
| 14.26 | 6.21 | 2.60 |
| 15.17 | 5.84 | 6.55 |
| 15.42 | 5.75 | 9.92 |
| 15.99 | 5.54 | 15.57 |
| 16.43 | 5.40 | 14.85 |
| 17.05 | 5.20 | 3.70 |
| 17.63 | 5.03 | 10.29 |
| 17.83 | 4.97 | 9.96 |
| 19.30 | 4.60 | 4.30 |
| 19.76 | 4.49 | 40.18 |
| 20.10 | 4.42 | 16.77 |
| 21.12 | 4.21 | 52.92 |
| 22.40 | 3.97 | 4.10 |
| 23.22 | 3.83 | 24.22 |
| 23.78 | 3.74 | 4.79 |
| 24.10 | 3.69 | 8.23 |
| 24.39 | 3.65 | 6.53 |
| 25.25 | 3.53 | 21.69 |
| 25.89 | 3.44 | 11.37 |
| 27.04 | 3.30 | 14.30 |
| 27.37 | 3.26 | 3.35 |
| 28.29 | 3.15 | 2.37 |
| 28.80 | 3.10 | 5.11 |
| 29.41 | 3.04 | 2.22 |

### Example 8: Preparation and XRPD Data of Compound a L-Malate Type a

Compound A free form type A (50 mg) was suspended in ethanol (0.50 mL) in a glass vial, followed by addition of L-malic acid (15 mg). The mixture was stirred at 50° C. using magnetic stirring for 2 h, then at 25° C. for 12 h. The resultant solid was separated to give Compound A as L-malate type A. The stoichiometric ratio of Compound A and malic acid is 1:1.

The L-malate salt Pattern A is hygroscopic. It absorbs about 7.9% water from 40%₀RH to 950% RiH at 25° C. No form change was observed after the DVS test.

The L-malate salt Type A was a hemihydrate of high crystallinity. DSC showed an endothermiic peak at Tₒₙₛₑₜ of 80.9° C., corresponding to dehydration. After that, a melting peak at Tₒₙₛₑₜ of 190.4° C. appeared. Decomposition occurred upon melting. TGA showed about 1.500 weight loss up to 121° C. No residual solvent was detected. KF analysis showed about 2.7% water by weight (0.67 equivalent by molar ratio). The L-malate salt Type A was chemically and physically stable at 25° C./92%₀RH in an open container, at 40° C./75%₀RH in an open container, and at 60° C. in a tight container over 1 week.

TABLE 8
Characteristic XRPD information of
Compound A L-malate type A

| Angle [°2θ] | d-spacing [Å] | Relative Intensity [%] |
|---|---|---|
| 5.06 | 17.46 | 36.50 |
| 6.97 | 12.66 | 21.00 |
| 7.28 | 12.14 | 9.50 |
| 10.09 | 8.76 | 29.20 |
| 10.49 | 8.42 | 17.20 |
| 13.36 | 6.62 | 61.30 |
| 14.67 | 6.03 | 46.10 |
| 15.13 | 5.85 | 100.00 |
| 16.08 | 5.51 | 68.00 |
| 17.02 | 5.21 | 38.70 |
| 18.10 | 4.90 | 15.00 |
| 18.44 | 4.81 | 27.90 |
| 18.74 | 4.73 | 19.20 |
| 19.54 | 4.54 | 8.30 |
| 20.05 | 4.42 | 30.60 |
| 20.41 | 4.35 | 57.50 |
| 21.07 | 4.21 | 23.20 |
| 22.35 | 3.98 | 36.50 |
| 22.82 | 3.89 | 40.20 |
| 23.45 | 3.79 | 48.00 |
| 23.83 | 3.73 | 33.3 |
| 25.36 | 3.51 | 73.80 |
| 25.72 | 3.46 | 41.10 |
| 28.14 | 3.17 | 21.10 |
| 29.55 | 3.02 | 19.70 |
| 30.57 | 2.92 | 11.70 |
| 31.22 | 2.86 | 7.30 |
| 32.36 | 2.76 | 4.80 |
| 33.38 | 2.68 | 3.40 |

### Example 9: Preparation and XRPD Data of Compound a Citrate Type a

Compound A free form type A (50 mg) was suspended in ethanol (0.50 mL) in a glass vial, followed by addition of citric acid (22 mg). The mixture was stirred at 50° C. using magnetic stirring for 2 h, then at 25° C. for 12 h. The resultant solid was separated to give Compound A as citrate type A. The stoichiometric ratio of Compound A and citric acid is 1:1.

The citrate salt Type A was an anhydrate of high crystallinity. DSC showed a melting peak at Tₒₙₛₑₜ of 212.9° C. Decomposition occurred upon melting. The TGA curve showed the weight loss of citrate salt type A from 30° C. to 100° C. is observed to be −0.243%. The citrate salt Type A was chemically and physically stable at 25° C./92%₀RH in an open container, at 40° C./75%₀RH in an open container, and at 60° C. in a tight container over 1 week. The citrate salt type A is slightly hygroscopic. It absorbs about 0.7% water from 0%₀RH to 95%₀RH at 25° C. No form change was observed after the DVS test. ¹H NMR (400 MHz, DMSO-d6) δ 12.36 (s, 1H), 8.90 (s, 1H), 8.50-8.43 (m, 1H), 8.24 (s, 1H), 8.22-8.16 (m, 1H), 7.91 (s, 1H), 7.88 (s, 1H), 7.24 (d, J=7.9 Hz, 1H), 4.04 (s, 2H), 3.26-3.15 (m, 2H), 3.15-3.07 (m, 2H), 3.03 (s, 3H), 2.83 (s, 3H), 2.79 (s, 3H), 2.63, 2.56 (AB q, J=15.2 Hz, 4H, citrate), 2.30 (s, 3H), 2.29 (s, 3H).

TABLE 9
Characteristic XRPD information of Compound A citrate type A

| Angle [°2θ] | d-spacing [Å] | Relative Intensity [%] |
|---|---|---|
| 4.95 | 17.84 | 4.00 |
| 6.59 | 13.41 | 6.80 |
| 9.03 | 9.79 | 34.10 |
| 9.98 | 8.86 | 10.80 |
| 10.84 | 8.16 | 13.00 |
| 13.02 | 6.80 | 6.20 |
| 13.23 | 6.69 | 7.70 |
| 14.56 | 6.08 | 8.50 |
| 14.96 | 5.92 | 100.00 |
| 15.55 | 5.70 | 25.30 |
| 15.94 | 5.56 | 6.70 |
| 16.71 | 5.30 | 3.50 |
| 17.56 | 5.05 | 84.30 |
| 19.22 | 4.62 | 3.90 |
| 20.09 | 4.42 | 3.20 |
| 20.60 | 4.31 | 19.60 |
| 21.59 | 4.11 | 3.40 |
| 21.96 | 4.04 | 6.00 |
| 22.33 | 3.98 | 53.30 |
| 22.52 | 3.94 | 38.50 |
| 23.26 | 3.82 | 10.70 |
| 24.17 | 3.68 | 4.90 |
| 24.41 | 3.64 | 6.20 |
| 25.11 | 3.54 | 5.90 |
| 26.08 | 3.41 | 64.00 |
| 26.88 | 3.31 | 3.80 |
| 27.43 | 3.25 | 4.30 |
| 27.92 | 3.19 | 4.80 |
| 30.09 | 2.97 | 10.00 |
| 30.75 | 2.91 | 9.20 |

### Example 10: Preparation and XRPD Data of Compound a Fumarate Type B

Compound A free form type A (50 mg) was suspended in acetone (0.50 mL) in a glass vial, followed by addition of fuimaric acid (7 mg). The mixture was stirred at 50° C. using magnetic stirring for 2 h, then at 25° C. for 12 h. The resultant solid was separated to give Compound A as fumarate type B. The stoichiometric ratio of Compound A and fumaric acid is 1:0.5.

TABLE 10
Characteristic XRPD information of Compound A fumarate type B

| Angle [°2θ] | d-spacing [Å] | Relative Intensity [%] |
|---|---|---|
| 4.83 | 18.29 | 1.90 |
| 6.66 | 13.27 | 3.00 |
| 8.44 | 10.47 | 3.40 |
| 9.22 | 9.58 | 2.00 |
| 10.69 | 8.27 | 5.80 |
| 11.60 | 7.62 | 9.40 |
| 12.06 | 7.33 | 31.30 |
| 12.80 | 6.91 | 18.30 |
| 13.44 | 6.58 | 28.10 |
| 13.86 | 6.39 | 4.70 |
| 14.29 | 6.19 | 2.40 |
| 15.43 | 5.73 | 9.70 |
| 16.02 | 5.53 | 28.30 |
| 16.33 | 5.42 | 3.20 |
| 16.95 | 5.22 | 29.20 |
| 17.54 | 5.05 | 2.60 |
| 18.18 | 4.87 | 3.60 |
| 18.45 | 4.80 | 6.80 |
| 18.97 | 4.67 | 11.50 |
| 19.75 | 4.49 | 1.80 |
| 20.03 | 4.43 | 1.40 |
| 20.57 | 4.31 | 10.10 |
| 21.13 | 4.20 | 18.00 |
| 21.48 | 4.13 | 6.10 |
| 22.30 | 3.98 | 10.30 |
| 23.15 | 3.84 | 3.40 |
| 23.96 | 3.71 | 8.40 |
| 24.67 | 3.61 | 34.90 |
| 24.98 | 3.56 | 100.00 |
| 26.91 | 3.31 | 4.20 |
| 27.85 | 3.20 | 2.00 |
| 28.46 | 3.13 | 4.40 |
| 31.01 | 2.88 | 3.10 |
| 31.45 | 2.84 | 2.10 |
| 35.59 | 2.52 | 3.10 |

### Example 11: Preparation and XRPD Data of Compound a Fumarate Type D

Compound A free form type A (50 mg) was suspended in tetrahydrofuran (0.50 mL) in a glass vial, followed by addition of fumaric acid (13 mg). The mixture was stirred at 50° C. using magnetic stirring for 2 h, then at 25° C. for 12 h. The resultant solid was separated to give Compound A as fumarate type D. The stoichiometric ratio of Compound A and fumaric acid is 1:1.

Compound A fumarate type D was medium crystallinity and the Melting onset was 160.5° C.

TABLE 11
Characteristic XRPD information of Compound A fumarate type D

| Angle [°2θ] | d-spacing [Å] | Relative Intensity [%] |
|---|---|---|
| 3.29 | 26.82 | 3.70 |
| 4.87 | 18.15 | 3.90 |
| 6.71 | 13.16 | 2.10 |
| 7.47 | 11.82 | 1.40 |
| 8.08 | 10.93 | 2.80 |
| 8.68 | 10.17 | 3.90 |
| 10.08 | 8.77 | 16.90 |
| 10.35 | 8.54 | 3.80 |
| 12.67 | 6.98 | 28.70 |
| 13.48 | 6.56 | 4.2 |
| 14.01 | 6.32 | 8.80 |
| 14.30 | 6.19 | 49.10 |
| 14.83 | 5.97 | 19.10 |
| 15.28 | 5.79 | 13.60 |
| 15.55 | 5.70 | 6.30 |
| 16.67 | 5.32 | 12.60 |
| 17.31 | 5.12 | 8.20 |
| 18.66 | 4.75 | 12.70 |
| 18.95 | 4.68 | 24.70 |
| 19.94 | 4.45 | 19.00 |
| 20.05 | 4.42 | 17.50 |
| 20.40 | 4.35 | 47.00 |
| 21.28 | 4.17 | 100.00 |
| 22.00 | 4.04 | 18.10 |
| 23.10 | 3.85 | 2.50 |
| 23.34 | 3.81 | 4.20 |
| 24.18 | 3.68 | 9.20 |
| 25.13 | 3.54 | 15.60 |
| 25.83 | 3.45 | 15.90 |
| 26.86 | 3.32 | 27.30 |
| 30.52 | 2.93 | 6.70 |
| 35.12 | 2.55 | 4.30 |
| 35.47 | 2.53 | 10.00 |

### Example 12: Preparation and XRPD Data of Compound a Maleate Type a

Compound A free form type A (50 mg) was suspended in acetonitrile (0.50 mL) in a glass vial, followed by addition of maleic acid (13 mg). The mixture was stirred at 50° C. using magnetic stirring for 2 h, then at 25° C. for 12 h. The resultant solid was separated to give Compound A as maleate type A. The stoichiometric ratio of Compound A and maleic acid is 1:1.

TABLE 12
Characteristic XRPD information of Compound A maleate type A

| Angle [°2θ] | d-spacing [Å] | Relative Intensity [%] |
|---|---|---|
| 5.07 | 17.42 | 54.20 |
| 8.06 | 10.97 | 26.80 |
| 8.81 | 10.04 | 12.40 |
| 11.70 | 7.56 | 8.20 |
| 12.71 | 6.96 | 18.70 |
| 13.44 | 6.59 | 97.30 |
| 14.77 | 5.99 | 38.60 |
| 15.25 | 5.81 | 22.60 |
| 15.51 | 5.71 | 19.10 |
| 16.18 | 5.47 | 86.00 |
| 16.44 | 5.39 | 43.60 |
| 17.32 | 5.12 | 21.10 |
| 17.56 | 5.05 | 23.10 |
| 19.02 | 4.66 | 30.60 |
| 19.43 | 4.57 | 29.40 |
| 20.92 | 4.24 | 95.90 |
| 21.38 | 4.15 | 40.10 |
| 22.20 | 4.00 | 38.90 |
| 22.69 | 3.92 | 6.20 |
| 23.48 | 3.79 | 11.50 |
| 24.03 | 3.70 | 20.50 |
| 24.80 | 3.59 | 17.60 |
| 25.23 | 3.53 | 100.00 |
| 25.99 | 3.43 | 7.90 |
| 26.91 | 3.31 | 45.00 |
| 27.37 | 3.26 | 26.90 |
| 27.99 | 3.19 | 13.20 |
| 29.49 | 3.03 | 11.90 |
| 31.39 | 2.85 | 8.20 |
| 32.33 | 2.77 | 6.60 |
| 33.13 | 2.70 | 12.30 |

### Example 13: Preparation and XRPD Data of Compound a Succinate Type B

Compound A free form type A (50 mg) was suspended in tetrahydrofuran (0.50 mL) in a glass vial, followed by addition of succinic acid (14 mg). The mixture was stirred at 50° C. using magnetic stirring for 2 h, then at 25° C. for 12 h. The resultant solid was separated to give Compound A as succinate type B with high crystallinity. The stoichiometric ratio of Compound A and succinic acid is 1:1.

TABLE 13
Characteristic XRPD information of Compound A succinate type B

| Angle [°2θ] | d-spacing [Å] | Relative Intensity [%] |
|---|---|---|
| 6.79 | 13.01 | 82.50 |
| 9.13 | 9.68 | 6.10 |
| 9.40 | 9.41 | 8.30 |
| 10.22 | 8.65 | 4.40 |
| 10.81 | 8.18 | 49.50 |
| 12.09 | 7.32 | 9.50 |
| 13.53 | 6.54 | 25.70 |
| 14.25 | 6.21 | 2.90 |
| 14.86 | 5.96 | 100.00 |
| 15.25 | 5.81 | 44.50 |
| 15.90 | 5.57 | 30.30 |
| 16.55 | 5.35 | 7.40 |
| 16.81 | 5.27 | 4.40 |
| 17.66 | 5.02 | 23.10 |
| 18.15 | 4.88 | 28.80 |
| 19.06 | 4.65 | 3.80 |
| 19.74 | 4.49 | 24.80 |
| 20.03 | 4.43 | 13.40 |
| 20.42 | 4.35 | 16.50 |
| 20.71 | 4.29 | 11.10 |
| 21.03 | 4.22 | 28.40 |
| 22.34 | 3.98 | 20.20 |
| 22.85 | 3.89 | 39.30 |
| 23.22 | 3.83 | 46.30 |
| 23.87 | 3.73 | 32.60 |
| 24.42 | 3.64 | 13.70 |
| 24.87 | 3.58 | 8.40 |
| 25.19 | 3.53 | 9.20 |
| 26.38 | 3.38 | 7.20 |
| 26.90 | 3.31 | 52.50 |
| 27.78 | 3.21 | 4.30 |
| 28.13 | 3.17 | 4.20 |
| 28.84 | 3.09 | 4.80 |
| 29.93 | 2.98 | 7.70 |

### Example 14: Preparation and XRPD Data of Compound a Hydrochloride Type a

Compound A free form type A (50 mg) was suspended in tetrahydrofuran (0.50 mL) in a glass vial, followed by addition of hydrochloric acid (115 L, 1.0 M). The mixture was stirred at 50° C. using magnetic stirring for 2 h, then at 25° C. for 12 h. The resultant solid was separated to give Compound A as hydrochloride type A. The stoichiometric ratio of Compound A and hydrogen chloride is 1:1.

TABLE 14
Characteristic XRPD information of Compound A hydrochloride type A

| Angle [°2θ] | d-spacing [Å] | Relative Intensity [%] |
|---|---|---|
| 5.55 | 15.92 | 65.20 |
| 8.12 | 10.88 | 11.80 |
| 8.41 | 10.51 | 8.50 |
| 9.29 | 9.51 | 14.90 |
| 11.83 | 7.48 | 6.20 |
| 12.08 | 7.32 | 7.40 |
| 13.47 | 6.57 | 100.00 |
| 15.42 | 5.74 | 22.10 |
| 15.75 | 5.62 | 27.10 |
| 16.16 | 5.48 | 6.60 |
| 16.53 | 5.36 | 12.00 |
| 16.94 | 5.23 | 11.10 |
| 18.00 | 4.92 | 27.20 |
| 18.60 | 4.77 | 14.90 |
| 19.84 | 4.47 | 25.60 |
| 20.24 | 4.38 | 9.20 |
| 21.72 | 4.09 | 18.20 |
| 22.13 | 4.01 | 29.40 |
| 23.08 | 3.85 | 40.70 |
| 23.55 | 3.77 | 44.40 |
| 24.44 | 3.64 | 43.70 |
| 26.15 | 3.40 | 47.70 |
| 26.38 | 3.38 | 79.20 |
| 26.91 | 3.31 | 7.00 |
| 27.92 | 3.19 | 15.60 |
| 28.32 | 3.15 | 9.40 |
| 33.12 | 2.70 | 13.60 |
| 33.27 | 2.69 | 11.30 |
| 34.17 | 2.62 | 11.00 |
| 35.28 | 2.54 | 15.90 |

### Example 15: Preparation and XRPD Data of Compound a Hydrochloride Type C

Compound A free form type A (50 mg) was suspended in acetonitrile (0.50 mL) in a glass vial, followed by addition of hydrochloric acid (115 μL, 1.0 M). The mixture was stirred at 50° C. using magnetic stirring for 2 h, then at 25° C. for 12 h. The resultant solid was separated to give Compound A as hydrochloride type C. The stoichiometric ratio of Compound A and hydrogen chloride is 1:1.

Hydrochloride salt type C was medium crystallinity. DSC showed the dehydration temperature onset was 53.4.

TABLE 15
Characteristic XRPD information of Compound A hydrochloride type C

| Angle [°2θ] | d-spacing [Å] | Relative Intensity [%] |
|---|---|---|
| 6.07 | 14.55 | 20.40 |
| 6.81 | 12.96262 | 100.00 |
| 8.31 | 10.64 | 2.70 |
| 9.80 | 9.02 | 3.30 |
| 12.08 | 7.32 | 12.40 |
| 12.85 | 6.88 | 11.80 |
| 13.17 | 6.72 | 24.00 |
| 13.55 | 6.53 | 45.50 |
| 13.89 | 6.37 | 43.40 |
| 15.71 | 5.64 | 12.60 |
| 16.11 | 5.50 | 10.80 |
| 16.68 | 5.31 | 8.40 |
| 18.12 | 4.89 | 39.30 |
| 18.72 | 4.74 | 3.60 |
| 19.34 | 4.59 | 12.80 |
| 20.31 | 4.37 | 23.70 |
| 20.86 | 4.26 | 12.90 |
| 22.17 | 4.01 | 15.10 |
| 23.89 | 3.72 | 9.00 |
| 25.07 | 3.55 | 12.90 |
| 25.44 | 3.50 | 70.80 |
| 26.05 | 3.42 | 15.40 |
| 26.46 | 3.37 | 45.30 |
| 27.12 | 3.29 | 37.00 |
| 27.48 | 3.24 | 27.80 |
| 28.06 | 3.18 | 23.20 |
| 28.77 | 3.10 | 9.00 |
| 29.13 | 3.06 | 12.70 |
| 29.79 | 3.00 | 2.50 |
| 30.40 | 2.94 | 9.60 |
| 30.71 | 2.9 | 4.70 |
| 31.97 | 2.80 | 4.10 |
| 33.75 | 2.65 | 3.90 |
| 35.28 | 2.54 | 7.00 |
| 35.74 | 2.51 | 4.50 |

### Example 16: Preparation and XRPD Data of Compound a Sulfate Type a

Compound A free form type A (50 mg) was suspended in acetonitrile (0.50 mL) in a glass vial, followed by addition of sulfuric acid (115 μL, 1.0 M). The mixture was stirred at 50° C. using magnetic stirring for 2 h, then at 25° C. for 12 h. The resultant solid was separated to give Compound A as sulfate type A. The stoichiometric ratio of Compound A and sulfuric acid is 1:0.5.

TABLE 16
Characteristic XRPD information of Compound A sulfate type A

| Angle [°2θ] | d-spacing [Å] | Relative Intensity [%] |
|---|---|---|
| 5.19 | 17.03 | 8.30 |
| 6.87 | 12.86 | 10.90 |
| 7.71 | 11.46 | 10.40 |
| 10.28 | 8.60 | 17.90 |
| 11.14 | 7.94 | 13.60 |
| 13.59 | 6.51 | 40.40 |
| 14.63 | 6.05 | 16.00 |
| 15.35 | 5.77 | 100.00 |
| 15.71 | 5.64 | 19.00 |
| 16.17 | 5.48 | 5.30 |
| 18.0 | 4.92 | 16.00 |
| 18.24 | 4.86 | 35.90 |
| 19.20 | 4.62 | 3.40 |
| 20.23 | 4.39 | 5.20 |
| 20.52 | 4.33 | 21.10 |
| 21.30 | 4.17 | 52.60 |
| 22.0 | 4.04 | 20.60 |
| 22.30 | 3.98 | 26.90 |
| 22.90 | 3.88 | 49.40 |
| 24.94 | 3.57 | 76.20 |
| 25.79 | 3.45 | 69.70 |
| 28.52 | 3.13 | 9.00 |
| 29.15 | 3.06 | 20.70 |
| 29.55 | 3.02 | 3.70 |

### Example 17: Preparation and XRPD Data of Compound a Citrate Type B

Compound A citrate type A (40 mg) was dissolved in a minimum amount of 2v:1v trifluoroethanol and acetone at 50° C. in a glass vial. The solution was filtered through a 0.45 m syringe filter. The filtrate was cooled to 5° C. at a rate of 0.1° C./min. The resultant solid was separated to give Compound A as citrate type B. The stoichiometric ratio of Compound A and citric acid is 1:1.

Citrate Type B was a potential trifluoroethanol channel solvate. It was obtained from trifluoroethanol/acetone (2:1, v/v) by slow cooling and fast cooling, and from TFE/ACN (2:1, v/v) by slow cooling. Wet cake of Citrate Type B had high crystallinity. DSC showed an endothermiic peak at Tₒₙₛₑₜ of 90.9° C. and an enthalpy of about 64 J/g, which corresponded to desolvation of TFE. After that, a melting peak at Tₒₙₛₑₜ of 197.5° C. appeared. Decomposition occurred upon melting. There were two weight loss steps in TGA, about 1.9% up to about 92° C. and about 8% from 92° C. to 164° C. After air dried overnight in fume hood, no residual solvent was detected by 1H-NMR. Stoichiometric ratio of free form to citric acid was 1:1. KF test shows it contains about 2.7% water by weight. Citrate Type B converted to Type A at ambient and open conditions (about 25° C. and about 80% RH) over 1 week.

TABLE 17
Characteristic XRPD information of Compound A citrate type B

| Angle [°2θ] | d-spacing [Å] | Relative Intensity [%] |
|---|---|---|
| 6.58 | 13.42 | 100.00 |
| 8.09 | 10.92 | 10.40 |
| 9.94 | 8.89 | 9.00 |
| 10.91 | 8.11 | 3.40 |
| 13.42 | 6.59 | 19.20 |
| 14.24 | 6.22 | 17.10 |
| 14.82 | 5.97 | 28.00 |
| 16.21 | 5.46 | 12.00 |
| 18.09 | 4.90 | 8.30 |
| 18.69 | 4.74 | 5.20 |
| 19.86 | 4.47 | 13.50 |
| 20.30 | 4.37 | 11.90 |
| 20.60 | 4.31 | 9.90 |
| 21.50 | 4.13 | 4.70 |
| 22.20 | 4.00 | 7.10 |
| 22.99 | 3.87 | 7.60 |

### Example 18: Preparation and XRPD Data of Compound a Citrate Type C

Compound A citrate type A (40 mg) was dissolved in minimum amount of 2v:1v trifluoroethanol and tetrahydrofuran at 50° C. in a glass vial. The solution was filtered through a 0.45 μM syringe filter. The filtrate was cooled to 5° C. at a rate of 0.1° C./min. The resultant solid was separated to give Compound A as citrate type C. The stoichiometric ratio of Compound A and citric acid is 4:3.

TABLE 18
Characteristic XRPD information of Compound A citrate type C

| Angle [°2θ] | d-spacing [Å] | Relative Intensity [%] |
|---|---|---|
| 4.26 | 20.73 | 100.00 |
| 5.40 | 16.35 | 18.00 |
| 6.02 | 14.67 | 5.60 |
| 6.61 | 13.37 | 2.30 |
| 8.47 | 10.43 | 39.10 |
| 10.80 | 8.19 | 9.90 |
| 12.05 | 7.34 | 3.60 |
| 12.70 | 6.96 | 43.00 |
| 15.52 | 5.71 | 7.10 |
| 16.96 | 5.22 | 8.20 |
| 19.55 | 4.54 | 13.00 |
| 21.24 | 4.18 | 27.30 |

### Example 19: Preparation and XRPD Data of Compound a Citrate Type E

Compound A citrate type A (40 mg) was dissolved in minimum amount of 2v:1v trifluoroethanol and acetonitrile at ambient temperature in a glass vial. The solution was filtered through a 0.45 μM syringe filter. The filtrate was left in a vial with a pierced cap for slow evaporation over a week. The resultant solid was separated to give Compound A as citrate type E. The stoichiometric ratio of Compound A and citric acid is 1:1.

TABLE 19
Characteristic XRPD information of Compound A citrate type E

| Angle [°2θ] | d-spacing [Å] | Relative Intensity [%] |
|---|---|---|
| 6.75 | 13.09 | 10.50% |
| 8.67 | 10.19 | 31.70% |
| 9.16 | 9.64 | 97.60% |
| 13.49 | 6.56 | 94.80% |
| 13.73 | 6.44 | 100.00% |
| 14.95 | 5.92 | 92.20% |
| 16.09 | 5.50 | 26.90% |
| 17.54 | 5.05 | 27.00% |
| 18.42 | 4.81 | 46.30% |
| 18.61 | 4.76 | 27.90% |
| 20.25 | 4.38 | 26.50% |
| 20.64 | 4.30 | 14.10% |
| 21.66 | 4.10 | 31.70% |
| 22.61 | 3.93 | 34.50% |
| 22.92 | 3.88 | 68.60% |
| 23.58 | 3.77 | 23.90% |
| 23.92 | 3.72 | 19.40% |
| 25.06 | 3.55 | 30.90% |
| 25.59 | 3.48 | 21.30% |
| 26.11 | 3.41 | 24.00% |
| 27.24 | 3.27 | 41.40% |
| 28.82 | 3.09 | 17.70% |
| 29.75 | 3.00 | 22.80% |
| 32.31 | 2.77 | 10.30% |
| 33.75 | 2.65 | 17.90% |

### Example 20: Preparation and XRPD Data of Compound a Citrate Type F

Compound A citrate type A (50 mg) was dissolved in dimethyl sulfoxide (1.0 mL) at ambient temperature in a glass vial. The solution was filtered through a 0.45 μM syringe filter. To the solution was slowly added water (4 mL), the resulting solution was mixed well then kept at 5° C. for a week. The resultant solid was separated to give Compound A as citrate type F. The stoichiometric ratio of Compound A and citric acid is 1:0.56.

TABLE 20
Characteristic XRPD information of Compound A citrate type F

| Angle [°2θ] | d-spacing [Å] | Relative Intensity [%] |
|---|---|---|
| 4.95 | 17.84 | 8.50 |
| 7.24 | 12.20 | 38.30 |
| 9.14 | 9.66 | 4.20 |
| 9.82 | 9.00 | 9.00 |
| 13.42 | 6.59 | 92.10 |
| 14.05 | 6.30 | 100.00 |
| 14.57 | 6.08 | 18.40 |
| 14.89 | 5.94 | 11.00 |
| 15.63 | 5.67 | 4.10 |
| 17.10 | 5.18 | 12.80 |
| 20.19 | 4.39 | 41.70 |
| 21.08 | 4.21 | 20.60 |
| 21.92 | 4.05 | 6.40 |

### Example 21: Pharmacokinetic Studies of Compound a Free Form Amorphous and Compound a Citrate Typea a in Mice

Compound A free form amorphous (115.7 mg) was prepared by lyophilizing a suspension of compound A free form in water with a minimum amount of acetonitrile.

Pharmacokinetic studies of Compound A were conducted in male CD-1 mice (Vital River, 8 weeks, 3 animals per dosing route) following Institutional Animal Care and Use Committee guidelines. Intravenous administration was formulated as 0.20 mg/mL solution in N,N-dimethylacetamide:30% solutol HS-15(w/v): saline=20:20:60 (V:V:V). Oral administration was formulated as 1.0 mg/mL suspension in 0.500 methyl cellulose. Blood samples were collected over a 24-h period post-dose. Plasma was isolated and processed with the below protocol: To an aliquot of 10 μL plasma was added 200 μL ACN containing 5 ng/mL terfenadine as internal standard. The mixture was vortexed for 1 m and centrifuged at 4000 rpm for 10 min. An aliquot of 50 μL supernatant was diluted with 150 μL acetonitrile/water. The concentration of Compound A was determined by LC-MS-MS. Non-compartmental pharmacokinetic analysis was performed on the concentration-time data.

TABLE 21
Pharmacokinetic parameters of Compound A in mice

| | | |
|---|---|---|
| | Compound A free | Compound A citrate |
| PK Parameters | form amorphous | type A |

IV Dose (1 mpk, calculated as free form)

| | | | |
|---|---|---|---|
| AUC₀₋₂₄ₕ | h · ng · mL⁻¹ | 344 | NA |

PO Dose (10 mpk, calculated as free form)

| | | | |
|---|---|---|---|
| t_{1/2} | h | 3.63 | 7.95 |
| tₘₐₓ | h | 3.50 | 1.50 |
| Cₘₐₓ | ng · mL⁻¹ | 177 | 640 |
| AUC₀₋₂₄ₕ | h · ng · mL⁻¹ | 1896 | 2990 |
| F % | | 55.2 | 86.9 |

### Example 22: Pharmacokinetic Studies of Compound a Free Form Amorphous and Compound a Citrate Type a in Rats

Pharmacokinetic studies of Compound A were conducted in male SD rats (Vital River, 8 weeks, 3 animals per dosing route) following Institutional Animal Care and Use Committee guidelines. Intravenous administration was formulated as 1.0 mg/mL solution in N,N-dimethylacetamide:30% solutol HS-15(w/v):saline=20:20:60 (V:V:V). Oral administration was formulated as 1.0 mg/mL suspension in 0.5% methyl cellulose. Blood samples were collected over a 24-h period post-dose. Plasma was isolated and processed with the below protocol: To an aliquot of 10 μL plasma was added 200 μL ACN containing 5 ng/mL terfenadine as internal standard. The mixture was vortexed for 1 min and centrifuged at 4000 rpm for 10 min. An aliquot of 50 μL supernatant was diluted with 150 μL acetonitrile/water. The concentration of Compound A was determined by LC-MS-MS. Non-compartmental pharmacokinetic analysis was performed on the concentration-time data.

TABLE 22
Pharmacokinetic parameters of Compound A in rats

| | | |
|---|---|---|
| | Compound A free | Compound A |
| PK Parameters | form amorphous | citrate type A |

IV Dose (1 mpk, calculated as free form)

| | | | |
|---|---|---|---|
| AUC₀₋₂₄ₕ | h · ng · mL⁻¹ | 656 | NA |

PO Dose (10 mpk, calculated as free form)

| | | | |
|---|---|---|---|
| t_{1/2} | h | 4.05 | NA |
| tₘₐₓ | h | 2.00 | 2.67 |
| Cₘₐₓ | ng · mL⁻¹ | 235 | 445 |
| AUC₀₋₂₄ₕ | h · ng · mL⁻¹ | 1124 | 1603 |
| F % | | 17.2 | 24.4 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

What is claimed is:

1. A pharmaceutically acceptable salt of 4-[2-(2,8-dimethyl-1,2,3,4-tetrahydroisoquinolin-6-yl)-5H-pyrrolo[2,3-b]pyrazin-7-yl]-N,N,2-trimethylbenzamide, wherein said pharmaceutically acceptable salts are inorganic salt(s) or organic salt(s).

2. The salt according to claim 1, which is in solid-state.

3. The salt according to claim 1 or 2, wherein the salt is an inorganic salt selected from hydrochloride, sulphate, phosphate, hydrobromide and/or nitrate; or is an organic salt selected from fumarate, tartrate (L-tartrate, D-tartrate or DL-tartrate), laurate, stearate, gentisate, nicotinate, aspartate (L-aspartate), succinate, adipate, malate (L-malate), citrate, maleate, glycolate, gluconate (D-gluconate), lactate (L-lactate), acetate, benzene sulfonate, methanesulfonate, mesylate, benzoate, naphthalene sulfonate, and/or oxalate;
preferably, the salt is selected from hydrochloride, sulphate, phosphate, hydrobromide, fumarate, tartrate (L-tartrate, D-tartrate or DL-tartrate), aspartate (L-aspartate), succinate, malate (L-malate), citrate, maleate, methanesulfonate or mesylate;
more preferably, the salt is selected from L-malate, citrate or succinate;
even more preferably, the salt is citrate.

4. The salt according to claim 3, wherein the salt is a compound of Formula (I): 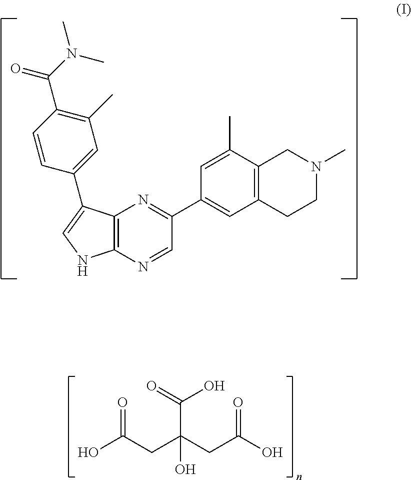
wherein n is a number from about 0.2 to about 2.0.

5. The salt according to claim 4, wherein n is a number about 0.3 to about 1.5; preferably n is a number selected from the group consisting of 0.3±0.1, 0.5±0.1, 0.7±0.1, 1.0±0.1 and 1.5±0.1;
preferably, n is a number selected from 0.3±0.05, 0.5±0.05, 0.6±0.05, 0.7±0.05, 0.8±0.05, 1.0±0.05, 1.1±0.05 and 1.5±0.05;
more preferably, n is 0.25˜0.35, 0.55˜0.65, 0.65˜0.75, 0.75˜0.85, 0.85˜0.95, 0.95˜1.05, 1.05˜1.15 or 1.45˜1.55;
even more preferably, n is about 0.3, 0.33, 0.40, 0.44, 0.45, 0.50, 0.55, 0.56, 0.60, 0.63, 0.65, 0.66, 0.67, 0.70, 0.75, 0.76, 0.80, 0.85, 0.90, 0.95, 0.96, 0.97, 0.98, 0.99, 1.0, 1.01, 1.02, 1.03, 1.04, 1.05, 1.06, 1.1, 1.45, 1.5, 1.55, 1.67, 1.8, 1.90, 1.95, 2.0.

6. The salt according to claim 3, wherein the salt is L-malate.

7. The salt according to claim 6, wherein the salt is a compound of Formula (II): 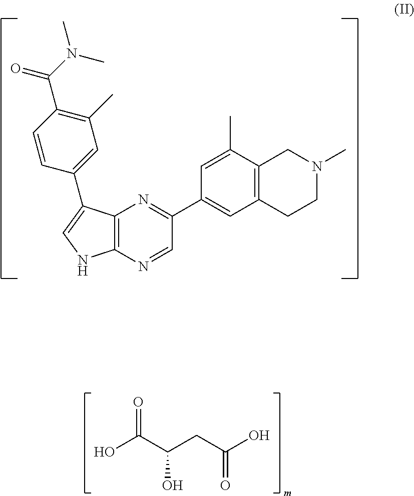
wherein m is a number from about 0.5 to about 2.0.

8. The salt according to claim 7, wherein m is a number about 0.5 to about 1.5;
preferably m is a number selected from the group consisting of 0.5±0.1, 0.7±0.1, 1.0±0.1 and 1.5±0.1;
more preferably, m is a number selected from 0.5±0.05, 0.6±0.05, 0.7±0.05, 0.8±0.05, 0.9±0.05, 1.0±0.05, 1.1±0.05, 1.2±0.05 and 1.5±0.05;
even more preferably, m is 0.95˜1.05, 1.05˜1.15, 1.15˜1.25 or 1.45˜1.55;
even more preferably, m is about 0.45, 0.50, 0.55, 0.95, 0.98, 0.99, 1.0, 1.01, 1.02, 1.05, 1.06, 1.07, 1.08, 1.09, 1.10, 1.11, 1.12, 1.13, 1.14, 1.15, 1.16, 1.17, 1.18, 1.19, 1.20, 1.3, 1.4, 1.45 or 1.5.

9. The salt according to claim 3, wherein the salt is succinate.

10. The salt according to claim 9, wherein the salt is a compound of Formula (III): 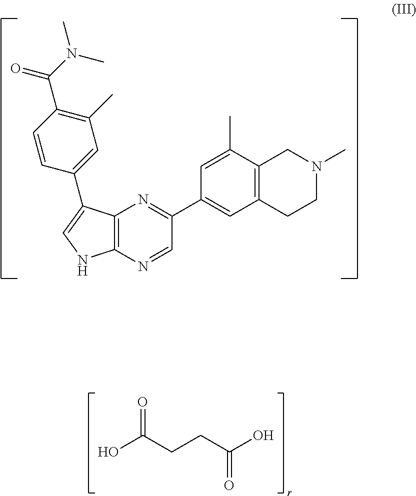
wherein r is a number from about 0.2 to about 2.0.

11. The salt according to claim 10, wherein r is a number about 0.5 to about 1.5;
preferably r is a number selected from the group consisting 0.5±0.1, 0.7±0.1, 1.0±0.1 and 1.5±0.1;
more preferably, r is a number selected from 0.5±0.05, 0.6±0.05, 0.7±0.05, 0.8±0.05, 0.9±0.05, 1.0±0.05, 1.1±0.05, 1.2±0.05 and 1.5±0.05;
even more preferably, r is 0.95˜1.05, 1.05˜1.15, 1.15˜1.25 or 1.45˜1.55;
even more preferably, r is 0.45, 0.50, 0.55, 0.95, 0.98, 0.99, 1.0, 1.01, 1.02, 1.05, 1.06, 1.07, 1.08, 1.09, 1.10, 1.11, 1.12, 1.13, 1.14, 1.15, 1.16, 1.17, 1.18, 1.19, 1.20, 1.3, 1.4, 1.45 or 1.5.

12. A pharmaceutical composition comprising a therapeutically effective amount of the salts according to any one of claims 1-11, and optionally one or more pharmaceutically acceptable carrier(s).

13. A method for treating or preventing a disorder or a disease selected from inflammatory disorder, autoimmune disease, or cancer, comprising administering a subject in need thereof a therapeutically effective amount of the salts according to any one of claims 1-11, or the pharmaceutical composition of claim 12.

14. A crystalline form of Formula IV 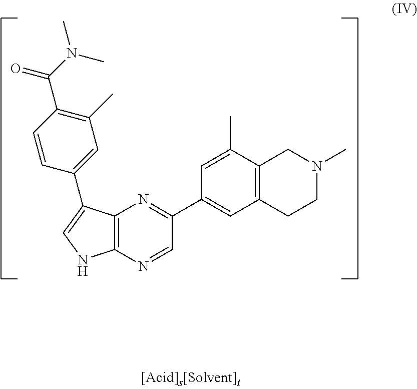
wherein [Acid] is selected from the group consisting of organic acids and inorganic acids;
[Solvent] is selected from H₂O or organic solvents;
s is a number from about 0.0 to about 5.0;
t is a number from about 0.0 to about 5.0.

15. A crystalline form of claim 14, wherein [Acid] is selected from the group consisting of hydrochloric acid, sulfuric acid, phosphoric acid, hydrobromic acid, nitric acid, fumaric acid, L-tartaric acid, D-tartaric acid, DL-tartaric acid, lauric acid, stearic acid, gentistic acid, nicotinic acid, aspartic acid, succinic acid, adipic acid, malic acid (L-malic acid), citric acid, maleic acid, ascorbic acid (L-ascorbic acid), glycolic acid, gluconic acid (D-gluconic acid), lactic acid (L-lactic acid), acetic acid, benzenesulfonic acid, methanesulfonic acid, benzoic acid, naphthalene sulfonic acid, and/or oxalic acid;
preferably [Acid] is selected from hydrochloric acid, sulfuric acid, phosphoric acid, hydrobromic acid, fumaric acid, tartaric acid (L-tartaric acid or D-tartaric acid), aspartic acid (L-aspartic acid), succinic acid, malic acid (L-malic acid), citric acid, maleic acid, methanesulfonic acid;
more preferably [Acid] is selected from L-malic acid, citric acid, succinic acid;
even more preferably [Acid] is selected from citric acid.

16. A crystalline form of any one of claims 14-15, wherein s is a number about 0 to about 1.5;
preferably s is a number selected from the group consisting 0.3±0.1, 0.5±0.1, 0.7±0.1, 1.0±0.1 and 1.5±0.1;
more preferably s is a number selected from the group consisting of 0.3±0.05, 0.5±0.05, 0.6±0.05, 0.7±0.05, 0.8±0.05, 1.0±0.05, 1.1±0.05, 1.2±0.05 and 1.5±0.05;
even more preferably, s is a number selected from the group consisting of 0.25˜0.35, 0.55˜0.65, 0.65˜0.75, 0.75˜0.85, 0.85˜0.95, 0.95˜1.05, 1.05˜1.15, 1.15˜1.25 or 1.45˜1.55;
even more preferably, s is 0.55˜0.65, 0.65˜0.75, 0.75˜0.85, 0.85˜0.95, 0.95˜1.05, 1.05˜1.15 or 1.45˜1.55; even more preferably, s is 0.3, 0.33, 0.40, 0.44, 0.45, 0.50, 0.55, 0.56, 0.60, 0.63, 0.65, 0.66, 0.67, 0.70, 0.75, 0.76, 0.80, 0.85, 0.90, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.20, 1.3, 1.4, 1.45, 1.5, 1.55, 1.67, 1.8, 1.90, 1.95, or 2.0.

17. A crystalline form of claim 14, wherein [Solvent] is selected from the group consisting of inorganic solvents selected from H₂O, MeOH, EtOH, n-PrOH, i-PrOH, 1-Butanol, sec-BuOH, tert-BuOH CF₃CH₂OH, acetone, toluene, THF, MeOAc, EtOAc, PrOAc, dioxane chloroform, DCM, butanone and MeCN or a combination of any of the foregoing;
preferably, [Solvent] is selected from the group consisting of inorganic solvents selected from H₂O, MeOH, EtOH, n-PrOH, i-PrOH, 1-BuOH, sec-BuOH, tert-BuOH, CF₃CH₂OH, acetone, toluene, THF, MeOAc, EtOAc, PrOAc, dioxane, chloroform, DCM, butanone, MeCN, (H₂O and EtOH), (H₂O and acetone) or (H₂O and MeCN);
even more preferably, solvents selected from H₂O, MeOH, EtOH, n-PrOH, i-PrOH, CF₃CH₂OH or (H₂O and EtOH) or (H₂O and MeOH) or (H₂O and i-PrOH) or (H₂O and n-PrOH) or (H₂O, n-PrOH and i-PrOH) or any combinations thereof.

18. A crystalline form of any one of claims 14 and 17, wherein t is a number about 0 to about 3;
preferably t is a number selected from the group consisting of 0, 0.5±0.2, 1.0±0.2, 1.5±0.2, 2.0±0.2, 2.5±0.2, 3.0±0.2;
more preferably, t is 0˜0.13, 0.13˜0.25, 0.25˜0.5, 0.5˜0.67, 0.67˜0.75, 0.75˜1, 1˜1.5, 1.5˜2, 2˜2.5, 2.5˜3;
even more preferably, t is about 0, 0.1, 0.14, 0.17, 0.2, 0.25, 0.3, 0.4, 0.45, 0.5, 0.50, 0.55, 0.57, 0.6, 0.65, 0.7, 0.8, 0.9, 0.95, 0.98, 1.02, 1.05, 1.08, 1.09, 1.0, 1.1, 1.11, 1.12, 1.2, 1.3, 1.4, 1.45, 1.5, 1.55, 1.6, 1.7, 1.8, 1.9, 2.0, 2.1, 2.2, 2.25, 2.3, 2.31, 2.4, 2.5, 2.6, 2.7, 2.8, 2.9, or 3.0.

19. A crystalline form of claim 14, wherein the crystalline form is free base and the crystalline form is Formula Va 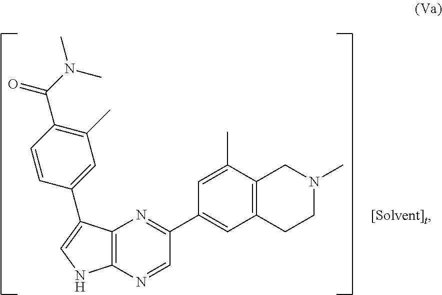
wherein [solvent] and t are as defined as claim 14;
preferably, the crystalline form is Formula Vb, Vc, Vd, Ve or Vf: 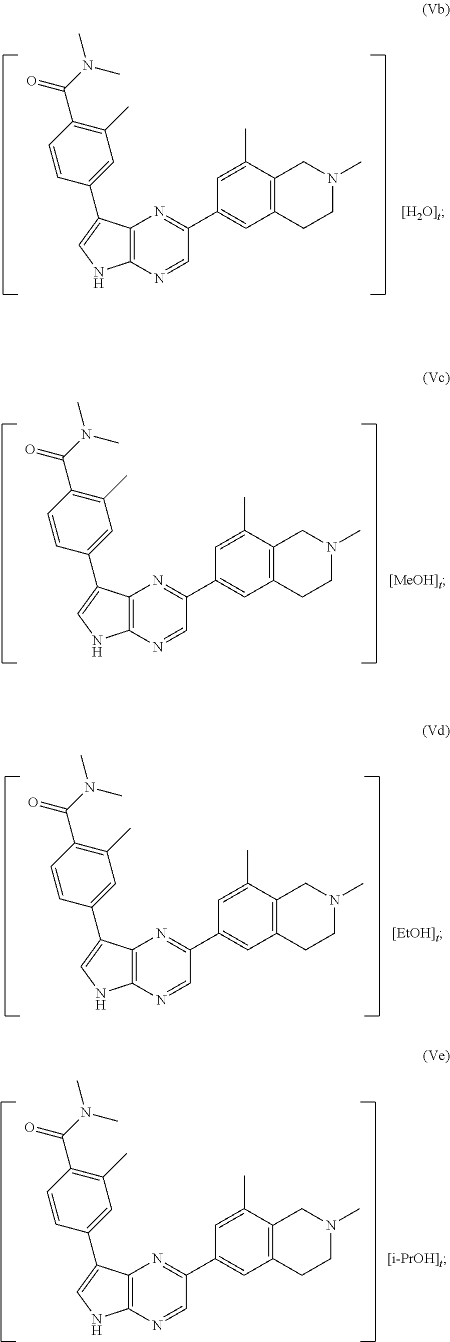 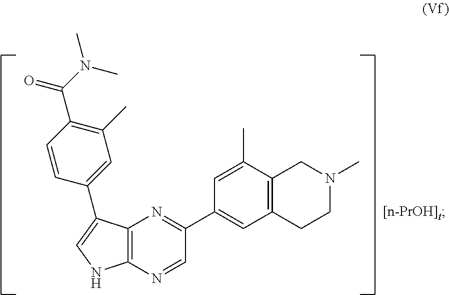
wherein t is as defined as claim 14 or 18;
preferably, t is about 0, 0.1, 0.14, 0.17, 0.2, 0.25, 0.3, 0.4, 0.45, 0.5, 0.50, 0.55, 0.57, 0.6, 0.65, 0.7, 0.8, 0.9, 0.95, 0.98, 1.02, 1.05, 1.08, 1.09, 1.0, 1.1, 1.11, 1.12, 1.2, 1.3, 1.4, 1.45, 1.5, 1.55, 1.6, 1.7, 1.8, 1.9, 2.0, 2.1, 2.2, 2.25, 2.3, 2.31, 2.4, 2.5, 2.6, 2.7, 2.8, 2.9, or 3.0.

20. The crystalline form of claim 19, which is selected from
Freeform Type A, characterized by a powder X-ray diffraction pattern comprising three, four, five, six, seven, eight, nine or more diffraction peaks having 2θ angle values independently selected from the group consisting of 9.10±0.2, 9.84±0.2, 10.61±0.2, 11.28±0.2, 12.87±0.2, 13.60±0.2, 14.65±0.2, 15.26±0.2, 15.93±0.2, 17.74±0.2, 18.41±0.2, 18.67±0.2, 19.12±0.2, 19.92±0.2, 21.30±0.2, 21.79±0.2, 22.85±0.2, 24.47±0.2, 26.01±0.2, 26.86±0.2 and 37.67±0.2 degrees; or
Freeform Type E, characterized by a powder X-ray diffraction pattern comprising three, four, five, six, seven, eight, nine or more diffraction peaks having 2θ angle values independently selected from the group consisting of 6.64±0.2, 8.65±0.2, 10.06±0.2, 10.94±0.2, 12.86±0.2, 13.84±0.2, 15.75±0.2, 16.40±0.2, 17.30±0.2, 18.00±0.2, 19.88±0.2, 20.44±0.2, 21.25±0.2, 21.94±0.2, 23.02±0.2, 23.38±0.2, 23.99±0.2, 25.14±0.2, 26.09±0.2, 26.73±0.2, 26.98±0.2 and 28.43±0.2 degrees; or
Freeform Type F, characterized by a powder X-ray diffraction pattern comprising three, four, five, six, seven, eight, nine or more diffraction peaks having 2θ angle values independently selected from the group consisting of 6.65±0.2, 7.14±0.2, 8.45±0.2, 10.88±0.2, 13.29±0.2, 13.46±0.2, 13.92±0.2, 14.46±0.2, 16.63±0.2, 16.96±0.2, 17.23±0.2, 17.57±0.2, 18.06±0.2, 18.98±0.2, 19.48±0.2, 19.66±0.2, 20.84±0.2, 21.66±0.2, 22.98±0.2, 23.33±0.2, 24.09±0.2, 24.35±0.2, 24.85±0.2, 25.53±0.2, 26.09±0.2, 26.72±0.2, 27.81±0.2, 28.31±0.2 and 28.79±0.2 degrees; or
Freeform Type I, characterized by a powder X-ray diffraction pattern comprising three, four, five, six, seven, eight, nine or more diffraction peaks having 2θ angle values independently selected from the group consisting of 5.84±0.2, 7.61±0.2, 9.11±0.2, 11.66±0.2, 13.61±0.2, 14.04±0.2, 14.35±0.2, 15.17±0.2, 15.85±0.2, 16.56±0.2, 17.35±0.2, 17.76±0.2, 18.30±0.2, 18.73±0.2, 19.23±0.2, 20.15±0.2, 20.67±0.2, 21.01±0.2, 21.58±0.2, 22.34±0.2, 23.79±0.2, 24.16±0.2, 24.90±0.2, 25.64±0.2, 26.06±0.2, 26.80±0.2 and 27.78±0.2 degrees; or
Freeform Type N, characterized by a powder X-ray diffraction pattern comprising three, four, five, six, seven, eight, nine or more diffraction peaks having 2θ angle values independently selected from the group consisting of 7.18±0.2, 7.78±0.2, 9.37±0.2, 14.38±0.2, 14.80±0.2, 15.47±0.2 and 21.64±0.2 degrees; or
Freeform Type W, characterized by a powder X-ray diffraction pattern comprising three, four, five, six, seven, eight, nine or more diffraction peaks having 2θ angle values independently selected from the group consisting of 6.62±0.2, 8.46±0.2, 11.11±0.2, 12.20±0.2, 13.24±0.2, 13.75±0.2, 14.26±0.2, 15.17±0.2, 15.42±0.2, 15.99±0.2, 16.43±0.2, 17.05±0.2, 17.63±0.2, 17.83±0.2, 19.30±0.2, 19.76±0.2, 20.10±0.2, 21.12±0.2, 22.40±0.2, 23.22±0.2, 23.78±0.2, 24.10±0.2, 24.39±0.2, 25.25±0.2, 25.89±0.2, 27.04±0.2, 27.37±0.2, 28.29±0.2, 28.80±0.2 and 29.41±0.2 degrees; or
Freeform Type Z, characterized by a powder X-ray diffraction pattern comprising three, four, five, six, seven, eight, nine or more diffraction peaks having 2θ angle values independently selected from the group consisting of 7.20±0.2, 10.06±0.2, 11.59±0.2, 13.72±0.2, 14.49±0.2, 15.85±0.2, 16.06±0.2, 17.38±0.2, 18.04±0.2, 19.60±0.2, 20.76±0.2, 21.56±0.2, 22.98±0.2, 23.49±0.2, 24.51±0.2, and 28.40±0.2 degrees.

21. The crystalline form of claim 19, which is selected from
Freeform Type A, characterized by a powder X-ray diffraction pattern comprising diffraction peaks having 2θ angle values of 5.33±0.2, 10.61±0.2, and 12.87±0.2 degrees; preferably having 20 angle values of 5.33±0.2, 10.61±0.2, 12.87±0.2, 15.93±0.2, and 26.01±0.2 degrees; more preferably having 2θ angle values of 5.33±0.2, 10.61±0.2, 12.87±0.2, 15.93±0.2, 19.12±0.2, 21.79±0.2, and 26.01±0.2 degrees; even more preferably having 2θ angle values of 5.33±0.2, 10.61±0.2, 12.87±0.2, 15.93±0.2, 18.41±0.2, 19.12±0.2, 21.30±0.2, 21.79±0.2, and 26.01±0.2 degrees; even more preferably having 2θ angle values of 5.33±0.2, 10.61±0.2, 12.87±0.2, 15.26±0.2, 15.93±0.2, 18.41±0.2, 18.67±0.2, 19.12±0.2, 21.30±0.2, 21.79±0.2, and 26.01±0.2 degrees; or
Freeform Type F, characterized by a powder X-ray diffraction pattern comprising diffraction peaks having 2θ angle values of 7.14±0.2, 8.45±0.2, and 24.35±0.2 degrees; preferably having 2θ angle values of 7.14±0.2, 8.45±0.2, 13.29±0.2, 16.63±0.2, and 24.35±0.2 degrees; more preferably having 2θ angle values of 7.14±0.2, 8.45±0.2, 13.29±0.2, 16.63±0.2, 16.96±0.2, 22.98±0.2, and 24.35±0.2 degrees; even more preferably having 2θ angle values of 7.14±0.2, 8.45±0.2, 13.29±0.2, 13.46±0.2, 13.92±0.2, 16.63±0.2, 16.96±0.2, 22.98±0.2, and 24.35±0.2 degrees; even more preferably having 2θ angle values of 6.65±0.2, 7.14±0.2, 8.45±0.2, 13.29±0.2, 13.46±0.2, 13.92±0.2, 16.63±0.2, 16.96±0.2, 22.98±0.2, 24.35±0.2, and 26.09±0.2 degrees; or
Freeform Type I, characterized by a powder X-ray diffraction pattern comprising diffraction peaks having 2θ angle values of 5.84±0.2, 16.56±0.2, and 25.64±0.2 degrees; preferably having 2θ angle values of 5.84±0.2, 14.04±0.2, 14.35±0.2, 16.56±0.2, and 25.64±0.2 degrees; more preferably having 20 angle values of 5.84±0.2, 14.04±0.2, 14.35±0.2, 15.85±0.2, 16.56±0.2, 25.64±0.2, and 26.06±0.2 degrees; even more preferably having 2θ angle values of 5.84±0.2, 7.61±0.2, 11.66±0.2, 14.04±0.2, 14.35±0.2, 15.85±0.2, 16.56±0.2, 25.64±0.2, and 26.06±0.2 degrees; even more preferably having 20 angle values of 5.84±0.2, 16.56±0.2, 25.64±0.2, 14.04±0.2, 14.35±0.2, 26.06±0.2, 15.85±0.2, 7.61±0.2, 11.66±0.2, 17.76±0.2, and 15.17±0.2 degrees.

22. A crystalline form of claim 14, wherein the crystalline form is Formula VIa 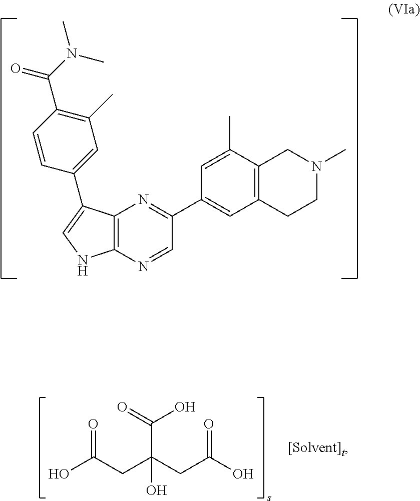
wherein [solvent], s and t are as defined as claim 14;
preferably, the crystalline form is Formula VIb, VIc, VId, VIe, VIf or VIg, 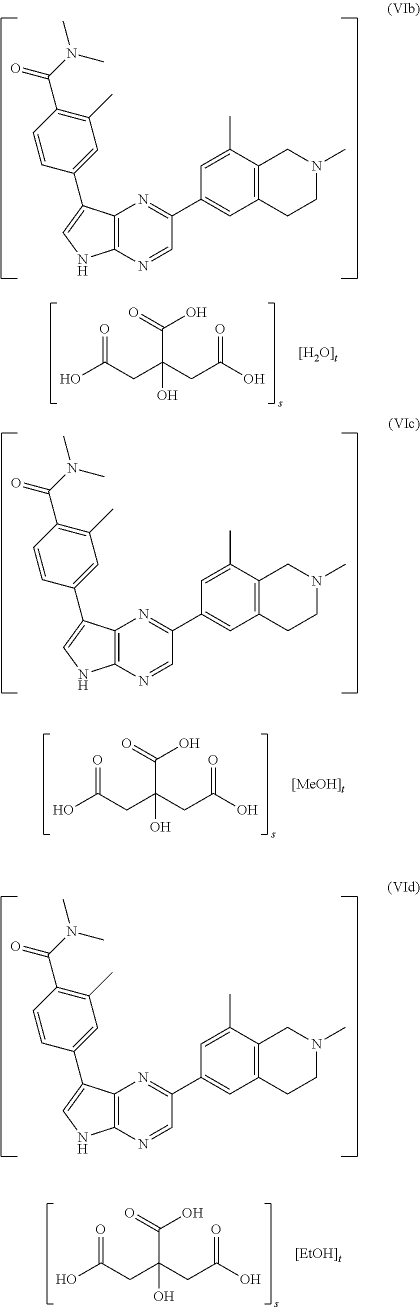 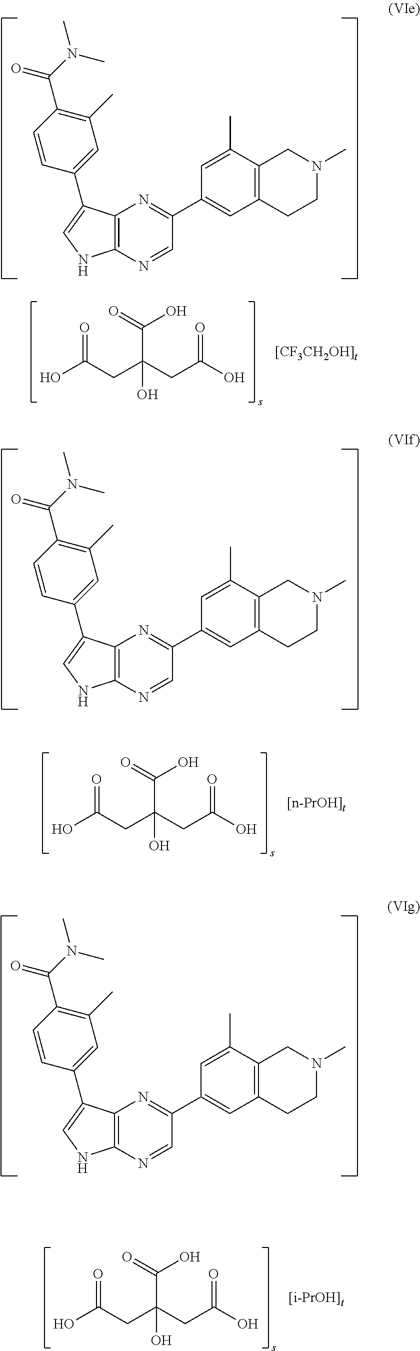
wherein s is about 0.3, 0.33, 0.40, 0.44, 0.45, 0.50, 0.55, 0.56, 0.60, 0.63, 0.65, 0.66, 0.67, 0.70, 0.75, 0.76, 0.80, 0.85, 0.90, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.20, 1.3, 1.4, 1.45, 1.5, 1.55, 1.67, 1.8, 1.90, 1.95, or 2.0; t is about 0, 0.1, 0.14, 0.17, 0.2, 0.25, 0.3, 0.4, 0.45, 0.5, 0.50, 0.55, 0.57, 0.6, 0.65, 0.7, 0.8, 0.9, 0.95, 0.98, 1.02, 1.05, 1.08, 1.09, 1.0, 1.1, 1.11, 1.12, 1.2, 1.3, 1.4, 1.45, 1.5, 1.55, 1.6, 1.7, 1.8, 1.9, 2.0, 2.1, 2.2, 2.25, 2.3, 2.31, 2.4, 2.5, 2.6, 2.7, 2.8, 2.9, or 3.0.

23. The crystalline form of claim 22, which is selected from
citrate salt Type A, characterized by a powder X-ray diffraction pattern comprising three, four, five, six, seven, eight, nine or more diffraction peaks having 2θ angle values independently selected from the group consisting of 4.95±0.2, 6.59±0.2, 9.03±0.2, 9.98±0.2, 10.84±0.2, 13.02±0.2, 13.23±0.2, 14.56±0.2, 14.96±0.2, 15.55±0.2, 15.94±0.2, 16.71±0.2, 17.56±0.2, 19.22±0.2, 20.09±0.2, 20.60±0.2, 21.59±0.2, 21.96±0.2, 22.33±0.2, 22.52±0.2, 23.26±0.2, 24.17±0.2, 24.41±0.2, 25.11±0.2, 26.08±0.2, 26.88±0.2, 27.43±0.2, 27.92±0.2, 30.09±0.2 and 30.75±0.2 degrees; or
citrate salt Type B, characterized by a powder X-ray diffraction pattern comprising three, four, five, six, seven, eight, nine or more diffraction peaks having 2θ angle values independently selected from the group consisting of 6.58±0.2, 8.09±0.2, 9.94±0.2, 10.91±0.2, 13.42±0.2, 14.24±0.2, 14.82±0.2, 16.21±0.2, 18.09±0.2, 18.69±0.2, 19.86±0.2, 20.30±0.2, 20.60±0.2, 21.50±0.2, 22.20±0.2 and 22.99±0.2 degrees; or
citrate salt Type C, characterized by a powder X-ray diffraction pattern comprising three, four, five, six, seven, eight, nine or more diffraction peaks having 2θ angle values independently selected from the group consisting of 4.26±0.2, 5.40±0.2, 6.02±0.2, 6.61±0.2, 8.47±0.2, 10.80±0.2, 12.05±0.2, 12.70±0.2, 15.52±0.2, 16.96±0.2, 19.55±0.2 and 21.24±0.2 degrees; or
citrate salt Type E, characterized by a powder X-ray diffraction pattern comprising three, four, five, six, seven, eight, nine or more diffraction peaks having 2θ angle values independently selected from the group consisting of 6.75±0.2, 8.67±0.2, 9.16±0.2, 13.49±0.2, 13.73±0.2, 14.95±0.2, 16.09±0.2, 17.54±0.2, 18.42±0.2, 18.61±0.2, 20.25±0.2, 20.64±0.2, 21.66±0.2, 22.61±0.2, 22.92±0.2, 23.58±0.2, 23.92±0.2, 25.06±0.2, 25.59±0.2, 26.11±0.2, 27.24±0.2, 28.82±0.2, 29.75±0.2, 32.31±0.2 and 33.75±0.2 degrees; or
citrate salt Type F, characterized by a powder X-ray diffraction pattern comprising three, four, five, six, seven, eight, nine or more diffraction peaks having 2θ angle values independently selected from the group consisting of 4.95±0.2, 7.24±0.2, 9.14±0.2, 9.82±0.2, 13.42±0.2, 14.05±0.2, 14.57±0.2, 14.89±0.2, 15.63±0.2, 17.10±0.2, 20.19±0.2, 21.08±0.2 and 21.92±0.2 degrees.

24. The crystalline form of claim 22, which is selected from
citrate salt Type A, characterized by a powder X-ray diffraction pattern comprising diffraction peaks having 2θ angle values of 14.96±0.2, 17.56±0.2 and 26.08±0.2 degrees; preferably having 20 angle values of 14.96±0.2, 17.56±0.2, 22.33±0.2, 22.52±0.2 and 26.08±0.2 degrees; more preferably having 2θ angle values of 9.03±0.2, 14.96±0.2, 15.55±0.2, 17.56±0.2, 22.33±0.2, 22.52±0.2 and 26.08±0.2 degrees; even more preferably having 2θ angle values of 9.03±0.2, 10.84±0.2, 14.96±0.2, 15.55±0.2, 17.56±0.2, 20.60±0.2, 22.33±0.2, 22.52±0.2 and 26.08±0.2 degrees; even more preferably having 2θ angle values of 9.03±0.2, 9.98±0.2, 10.84±0.2, 14.96±0.2, 15.55±0.2, 17.56±0.2, 20.60±0.2, 22.33±0.2, 22.52±0.2, 23.26±0.2 and 26.08±0.2 degrees; or
citrate salt Type B, characterized by a powder X-ray diffraction pattern comprising diffraction peaks having 2θ angle values of 6.58±0.2, 13.42±0.2, and 14.82±0.2 degrees; preferably having 20 angle values of 6.58±0.2, 13.42±0.2, 14.24±0.2, 14.82±0.2, and 19.86±0.2 degrees; more preferably having 2θ angle values of 6.58±0.2, 13.42±0.2, 14.24±0.2, 14.82±0.2, 16.21±0.2, 19.86±0.2, and 20.30±0.2 degrees; even more preferably having 2θ angle values of 6.58±0.2, 8.09±0.2, 13.42±0.2, 14.24±0.2, 14.82±0.2, 16.21±0.2, 19.86±0.2, 20.30±0.2, and 20.60±0.2; degrees even more preferably having 2θ angle values of 6.58±0.2, 8.09±0.2, 9.94±0.2, 13.42±0.2, 14.24±0.2, 14.82±0.2, 16.21±0.2, 18.09±0.2, 19.86±0.2, 20.30±0.2, and 20.60±0.2 degrees; or
citrate salt Type C, characterized by a powder X-ray diffraction pattern comprising diffraction peaks having 2θ angle values of 4.26±0.2, 8.47±0.2, and 12.70±0.2 degrees; preferably having 2θ angle values of 4.26±0.2, 5.40±0.2, 8.47±0.2, 12.70±0.2, and 21.24±0.2 degrees; more preferably having 20 angle values of 4.26±0.2, 5.40±0.2, 8.47±0.2, 10.80±0.2, 12.70±0.2, 21.24±0.2, and 19.55±0.2 degrees; even more preferably having 2θ angle values of 4.26±0.2, 5.40±0.2, 8.47±0.2, 10.80±0.2, 12.70±0.2, 15.52±0.2, 16.96±0.2, 19.55±0.2, and 21.24±0.2 degrees; even more preferably having 2θ angle values of 4.26±0.2, 5.40±0.2, 6.02±0.2, 8.47±0.2, 10.80±0.2, 12.70±0.2, 15.52±0.2, 16.96±0.2, 19.55±0.2, 19.91±0.2, and 21.24±0.2 degrees; or
citrate salt Type E, characterized by a powder X-ray diffraction pattern comprising diffraction peaks having 2θ angle values of 9.16±0.2, 13.49±0.2, and 13.73±0.2 degrees; preferably having 20 angle values of 9.16±0.2, 13.49±0.2, 13.73±0.2, 14.95±0.2, and 22.92±0.2 degrees; more preferably having 2θ angle values of 9.16±0.2, 13.49±0.2, 13.73±0.2, 14.95±0.2, 18.42±0.2, 22.92±0.2, and 27.24±0.2 degrees; even more preferably having 2θ angle values of 8.67±0.2, 9.16±0.2, 13.49±0.2, 13.73±0.2, 14.95±0.2, 18.42±0.2, 22.61±0.2, 22.92±0.2, and 27.24±0.2 degrees; even more preferably having 2θ angle values of 8.67±0.2, 9.16±0.2, 13.49±0.2, 13.73±0.2, 14.95±0.2, 18.42±0.2, 21.66±0.2, 22.61±0.2, 22.92±0.2, 25.06±0.2, and 27.24±0.2 degrees; or
citrate salt Type F, characterized by a powder X-ray diffraction pattern comprising diffraction peaks having 2θ angle values of 13.42±0.2, 14.05±0.2, and 20.19±0.2 degrees; preferably having 20 angle values of 7.24±0.2, 13.42±0.2, 14.05±0.2, 20.19±0.2, and 21.08±0.2 degrees; more preferably having 2θ angle values of 7.24±0.2, 13.42±0.2, 14.05±0.2, 14.57±0.2, 17.10±0.2, 20.19±0.2, and 21.08±0.2 degrees; even more preferably having 2θ angle values of 7.24±0.2, 9.82±0.2, 13.42±0.2, 14.05±0.2, 14.57±0.2, 14.89±0.2, 17.10±0.2, 20.19±0.2 and 21.08±0.2 degrees; even more preferably having 2θ angle values of 4.95±0.2, 7.24±0.2, 9.82±0.2, 13.42±0.2, 14.05±0.2, 14.57±0.2, 14.89±0.2, 17.10±0.2, 20.19±0.2, 21.08±0.2 and 21.92±0.2 degrees.

25. The crystalline form of claim 14, which is selected from
L-malate salt Type A, characterized by a powder X-ray diffraction pattern comprising three, four, five, six, seven, eight, nine or more diffraction peaks having 2θ angle values independently selected from the group consisting of 5.06±0.2, ±0.2, 6.97±0.2, 7.28±0.2, 10.09±0.2, 10.49±0.2, 13.36±0.2, 14.67±0.2, 15.13±0.2, 16.08±0.2, 17.02±0.2, 18.10±0.2, 18.44±0.2, 18.74±0.2, 19.54±0.2, 20.05±0.2, 20.41±0.2, 21.07±0.2, 22.35±0.2, 22.82±0.2, 23.45±0.2, 23.83±0.2, 25.36±0.2, 25.72±0.2, 28.14±0.2, 29.55±0.2, 30.57±0.2, 31.22±0.2, 32.36±0.2 and 33.38±0.2 degrees; or
succinate salt Type B, characterized by a powder X-ray diffraction pattern comprising three, four, five, six, seven, eight, nine or more diffraction peaks having 2θ angle values independently selected from the group consisting of 6.79±0.2, 9.13±0.2, 9.40±0.2, 10.22±0.2, 10.81±0.2, 12.09±0.2, 13.53±0.2, 14.25±0.2, 14.86±0.2, 15.25±0.2, 15.90±0.2, 16.55±0.2, 16.81±0.2, 17.66±0.2, 18.15±0.2, 19.06±0.2, 19.74±0.2, 20.03±0.2, 20.42±0.2, 20.71±0.2, 21.03±0.2, 22.34±0.2, 22.85±0.2, 23.22±0.2, 23.87±0.2, 24.42±0.2, 24.87±0.2, 25.19±0.2, 26.38±0.2, 26.90±0.2, 27.78±0.2, 28.13±0.2, 28.84±0.2 and 29.93±0.2 degrees; or
fumarate Type B, characterized by a powder X-ray diffraction pattern comprising three, four, five, six, seven, eight, nine or more diffraction peaks having 2θ angle values independently selected from the group consisting of 4.83±0.2, 6.66±0.2, 8.44±0.2, 9.22±0.2, 10.69±0.2, 11.60±0.2, 12.06±0.2, 12.80±0.2, 13.44±0.2, 13.86±0.2, 14.29±0.2, 15.44±0.2, 16.02±0.2, 16.33±0.2, 16.95±0.2, 17.54±0.2, 18.18±0.2, 18.46±0.2, 18.97±0.2, 19.75±0.2, 20.03±0.2, 20.57±0.2, 21.14±0.2, 21.48±0.2, 22.30±0.2, 23.15±0.2, 23.96±0.2, 24.67±0.2, 24.98±0.2, 26.91±0.2, 27.85±0.2, 28.46±0.2, 31.01±0.2, 31.45±0.2, and 35.59±0.2 degrees; or
fumarate Type D, characterized by a powder X-ray diffraction pattern comprising three, four, five, six, seven, eight, nine or more diffraction peaks having 2θ angle values independently selected from the group consisting of 3.29±0.2, 4.87±0.2, 6.71±0.2, 7.47±0.2, 8.08±0.2, 8.68±0.2, 10.08±0.2, 10.35±0.2, 12.67±0.2, 13.48±0.2, 14.01±0.2, 14.30±0.2, 14.83±0.2, 15.28±0.2, 15.55±0.2, 16.67±0.2, 17.31±0.2, 18.66±0.2, 18.95±0.2, 19.94±0.2, 20.05±0.2, 20.40±0.2, 21.28±0.2, 22.00±0.2, 23.10±0.2, 23.34±0.2, 24.18±0.2, 25.13±0.2, 25.83±0.2, 26.86±0.2, 30.52±0.2, 35.12±0.2 and 35.47±0.2 degrees; or
maleate Type A, characterized by a powder X-ray diffraction pattern comprising three, four, five, six, seven, eight, nine or more diffraction peaks having 2θ angle values independently selected from the group consisting of 5.07±0.2, 8.06±0.2, 8.81±0.2, 11.70±0.2, 12.71±0.2, 13.44±0.2, 14.77±0.2, 15.25±0.2, 15.51±0.2, 16.18±0.2, 16.44±0.2, 17.32±0.2, 17.56±0.2, 19.02±0.2, 19.43±0.2, 20.92±0.2, 21.38±0.2, 22.20±0.2, 22.69±0.2, 23.48±0.2, 24.03±0.2, 24.80±0.2, 25.23±0.2, 25.99±0.2, 26.91±0.2, 27.37±0.2, 27.99±0.2, 29.49±0.2, 31.39±0.2, 32.33±0.2 and 33.13±0.2 degrees; or
hydrochloride Type A, characterized by a powder X-ray diffraction pattern comprising three, four, five, six, seven, eight, nine or more diffraction peaks having 2θ angle values independently selected from the group consisting of 5.55±0.2, 8.12±0.2, 8.41±0.2, 9.29±0.2, 11.83±0.2, 12.08±0.2, 13.47±0.2, 15.42±0.2, 15.75±0.2, 16.16±0.2, 16.53±0.2, 16.94±0.2, 18.00±0.2, 18.60±0.2, 19.84±0.2, 20.24±0.2, 21.72±0.2, 22.13±0.2, 23.08±0.2, 23.55±0.2, 24.44±0.2, 26.15±0.2, 26.38±0.2, 26.91±0.2, 27.92±0.2, 28.32±0.2, 33.12±0.2, 33.27±0.2, 34.17±0.2 and 35.28±0.2 degrees; or
hydrochloride Type C, characterized by a powder X-ray diffraction pattern comprising three, four, five, six, seven, eight, nine or more diffraction peaks having 2θ angle values independently selected from the group consisting of 6.07±0.2, 6.81±0.2, 8.31±0.2, 9.80±0.2, 12.08±0.2, 12.85±0.2, 13.17±0.2, 13.55±0.2, 13.89±0.2, 15.71±0.2, 16.11±0.2, 16.68±0.2, 18.12±0.2, 18.72±0.2, 19.34±0.2, 20.31±0.2, 20.86±0.2, 22.17±0.2, 23.89±0.2, 25.07±0.2, 25.44±0.2, 26.05±0.2, 26.46±0.2, 27.12±0.2, 27.48±0.2, 28.06±0.2, 28.77±0.2, 29.13±0.2, 29.79±0.2, 30.40±0.2, 30.71±0.2, 31.97±0.2, 33.75±0.2, 35.28±0.2 and 35.74±0.2 degrees; or
sulfate Type A, characterized by a powder X-ray diffraction pattern comprising three, four, five, six, seven, eight, nine or more diffraction peaks having 2θ angle values independently selected from the group consisting of 5.19±0.2, 6.87±0.2, 7.71±0.2, 10.28±0.2, 11.14±0.2, 13.59±0.2, 14.63±0.2, 15.35±0.2, 15.71±0.2, 16.17±0.2, 18.00±0.2, 18.24±0.2, 19.20±0.2, 20.23±0.2, 20.52±0.2, 21.30±0.2, 22.00±0.2, 22.30±0.2, 22.90±0.2, 24.94±0.2, 25.79±0.2, 28.52±0.2, 29.15±0.2 and 29.55±0.2 degrees.

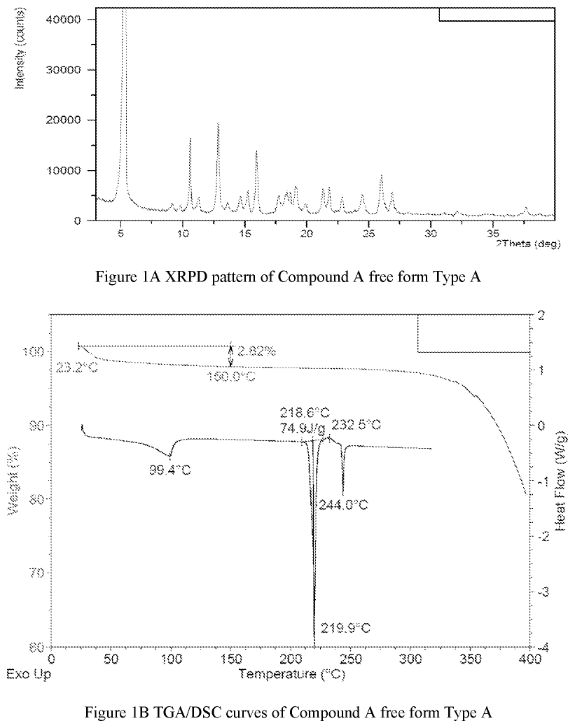

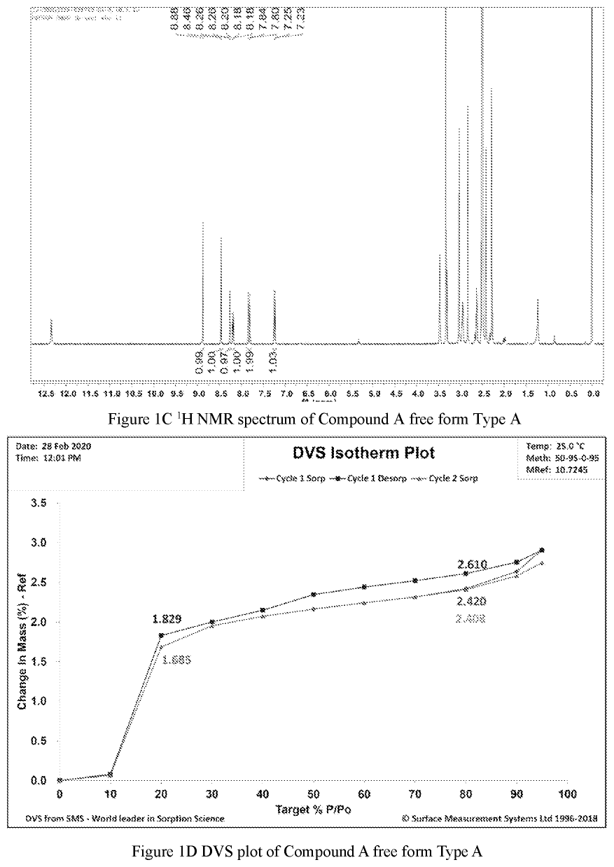

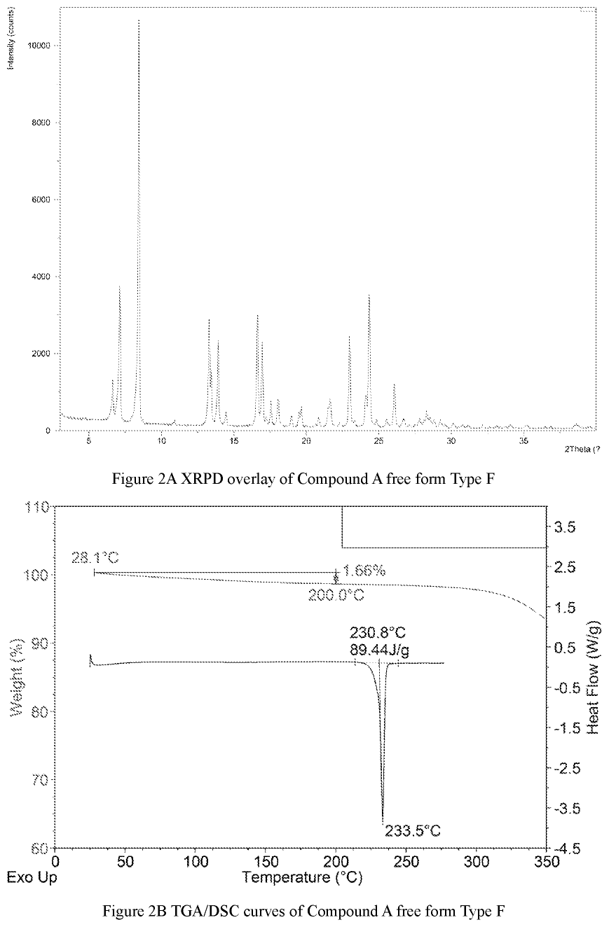

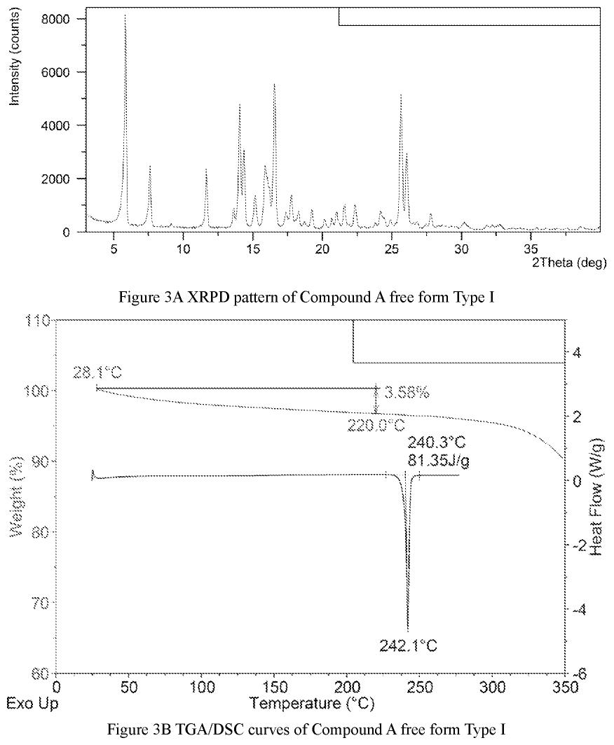

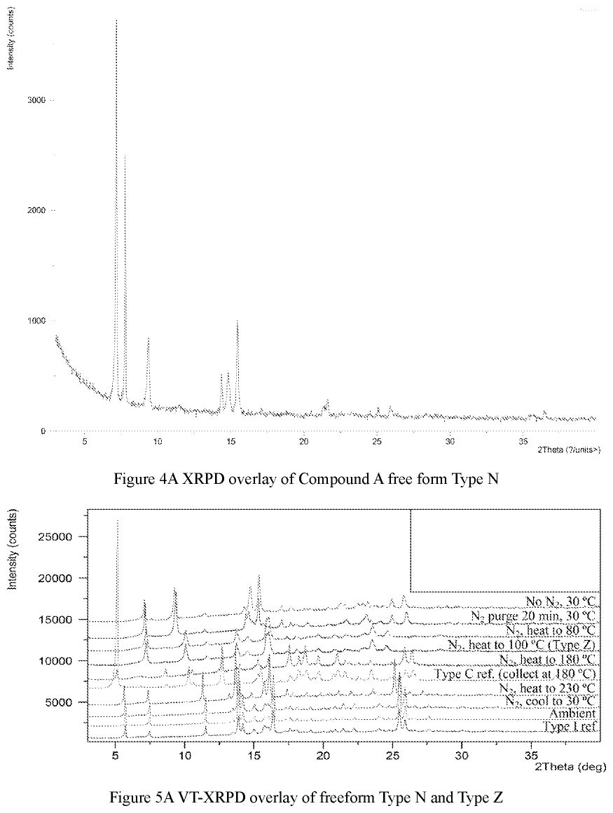

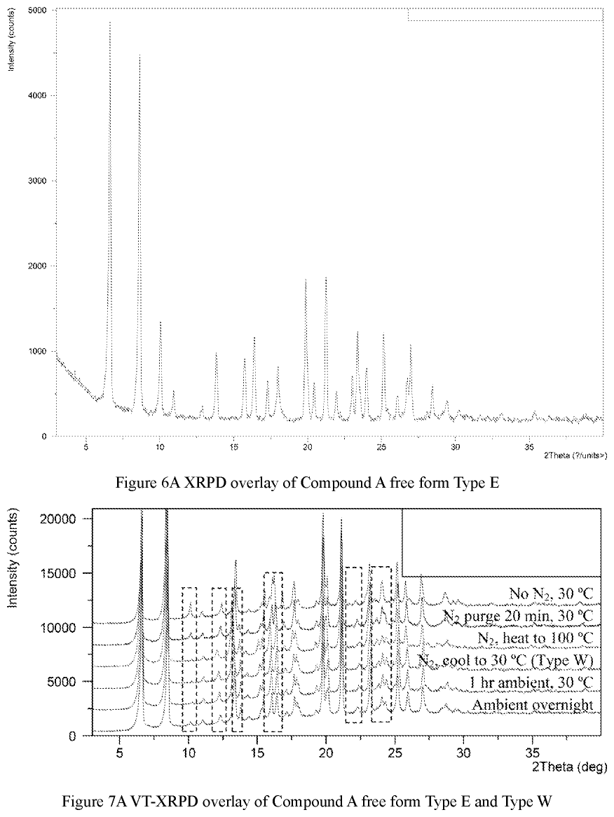

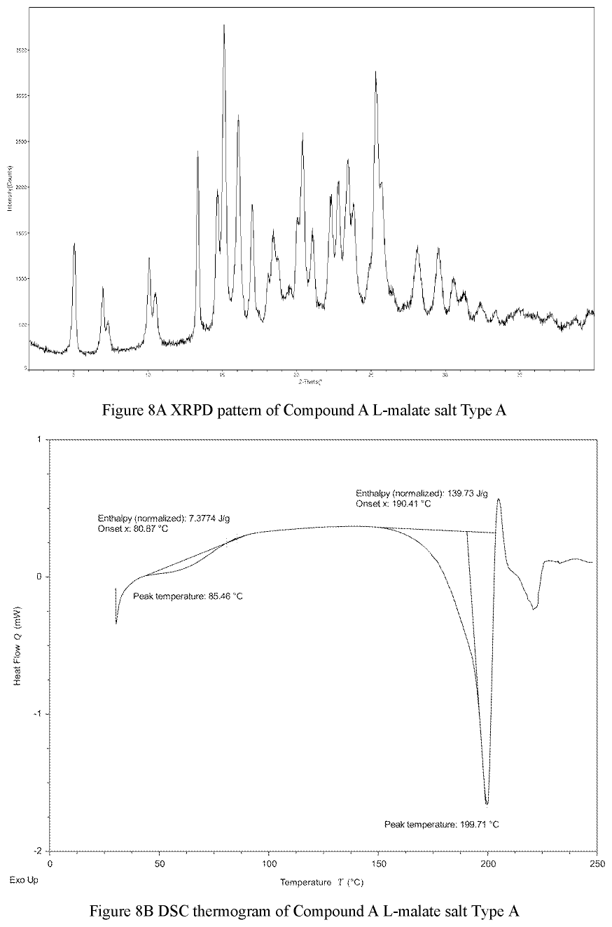

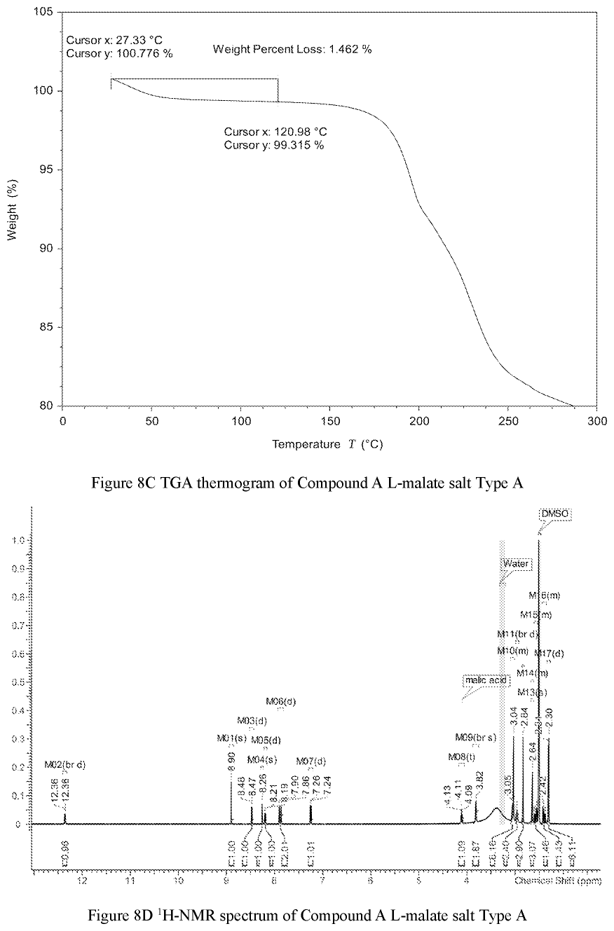

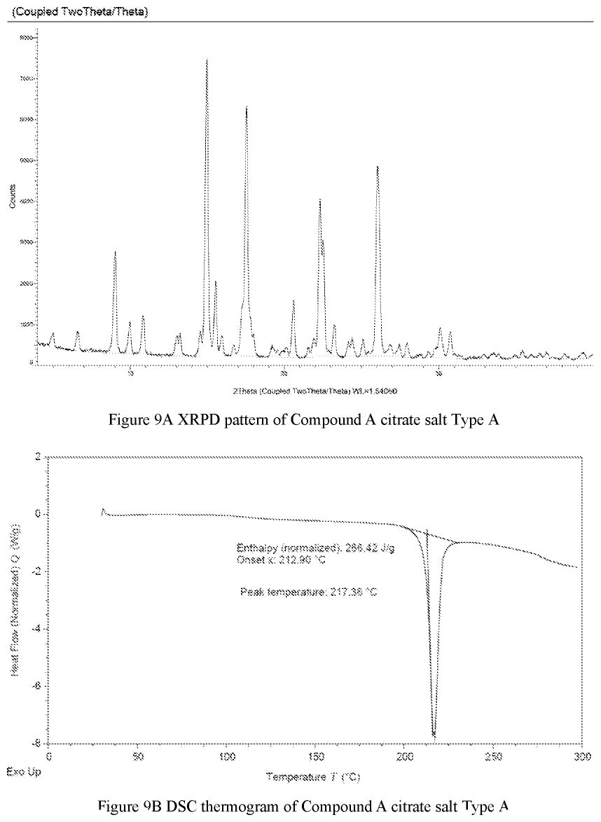

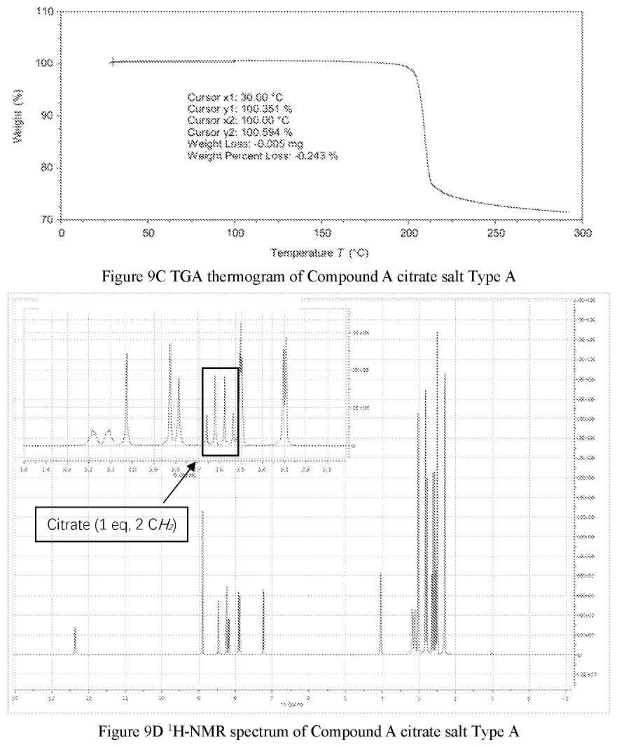

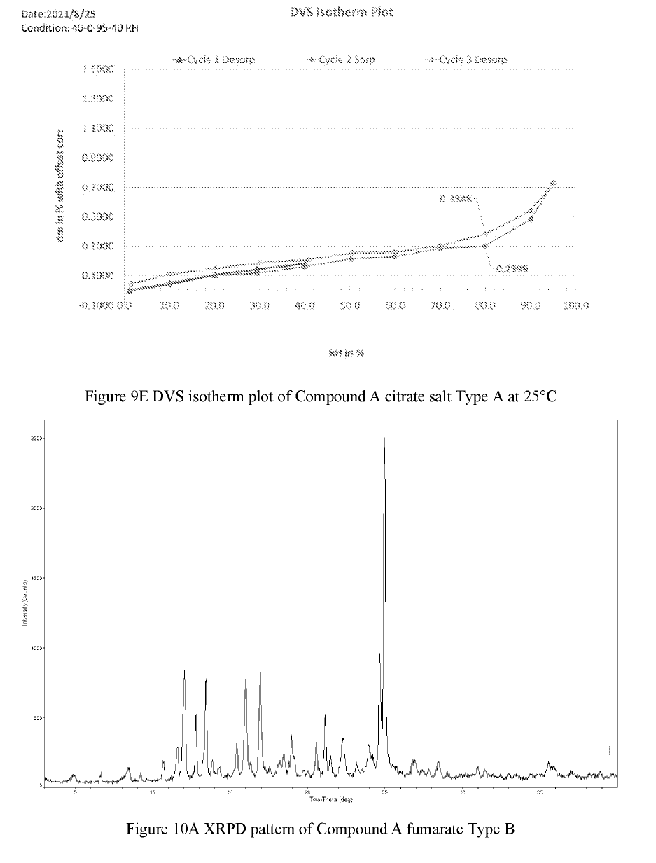

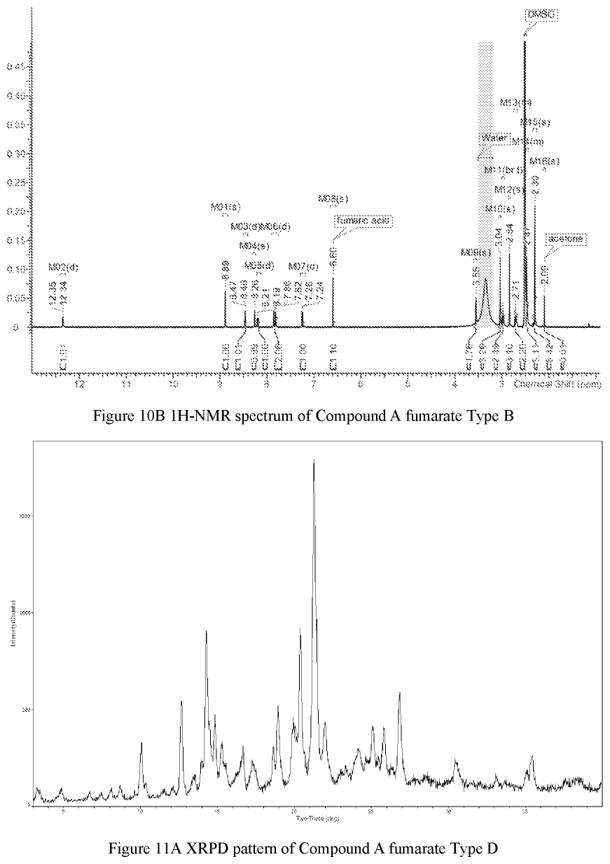

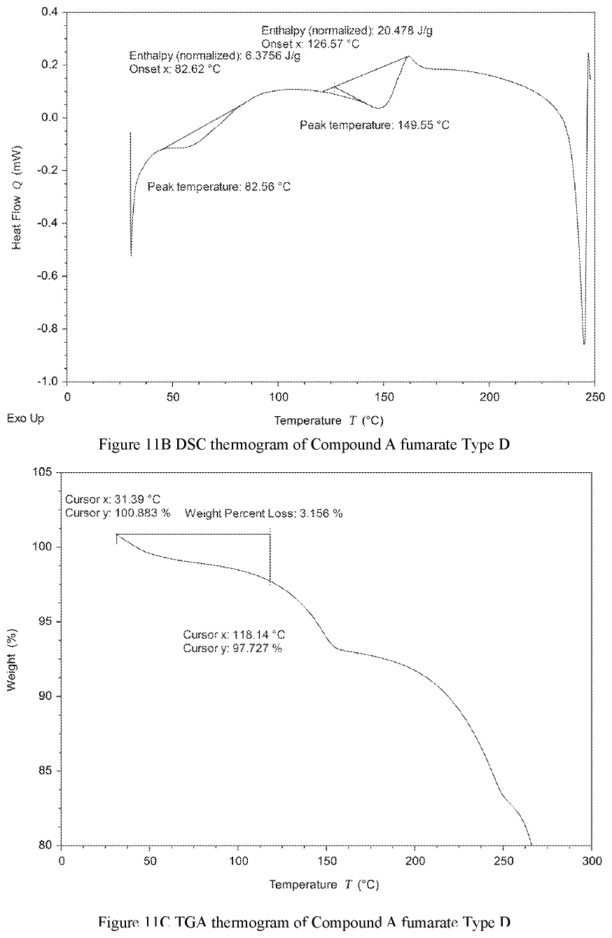

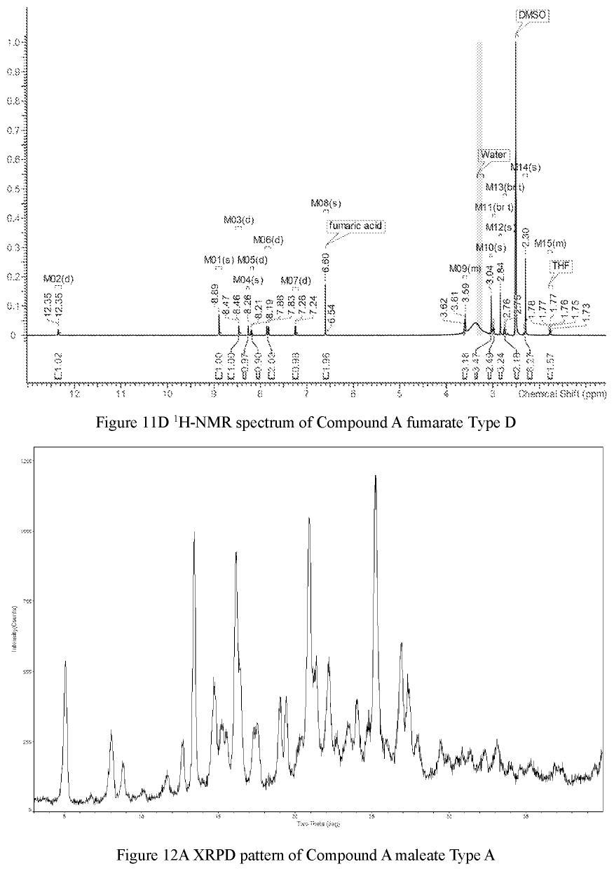

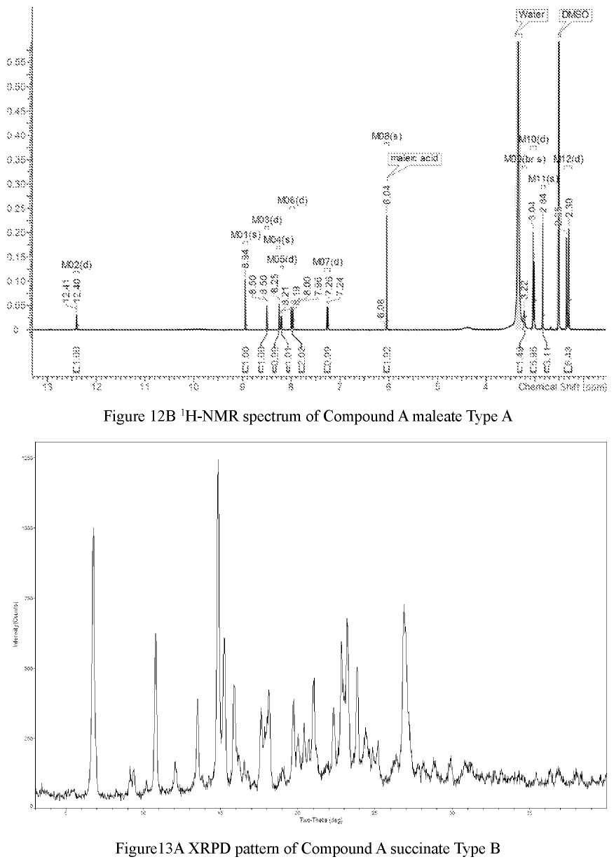

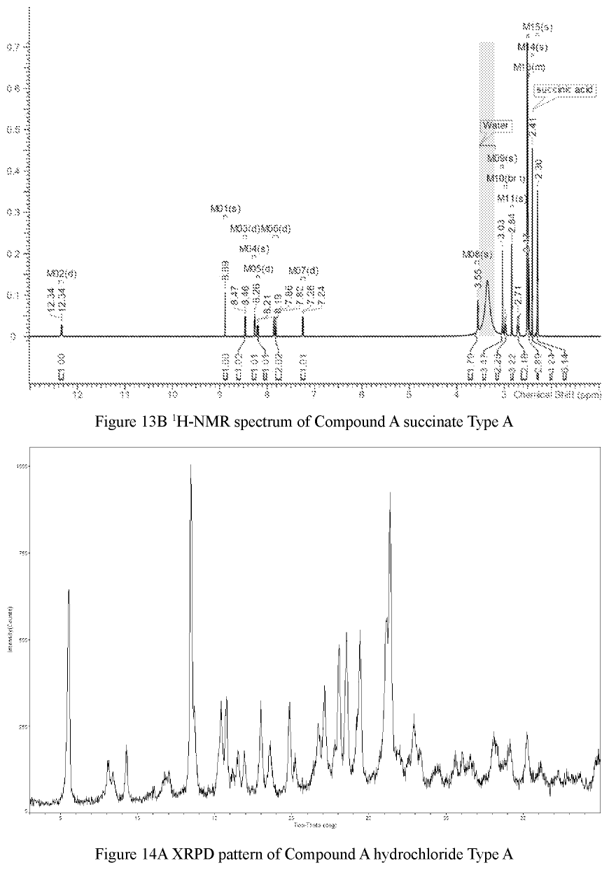

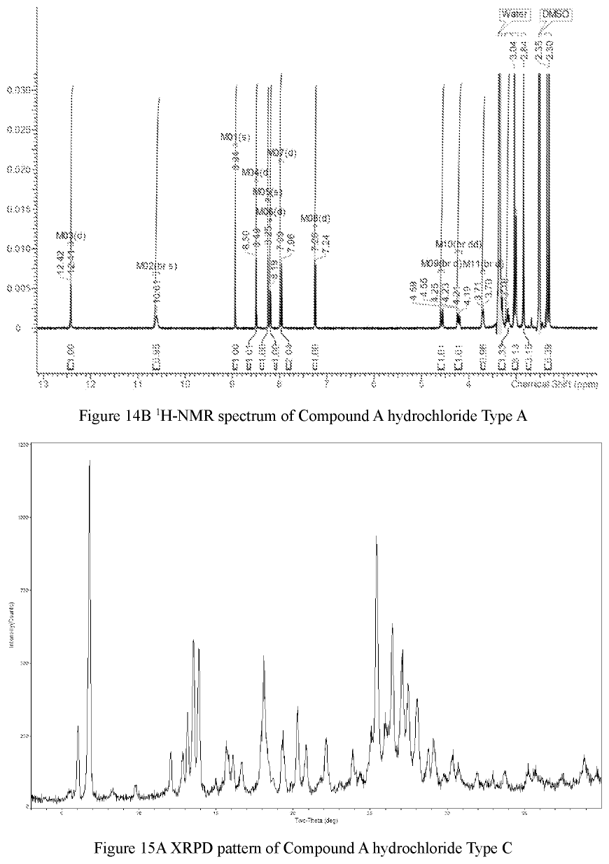

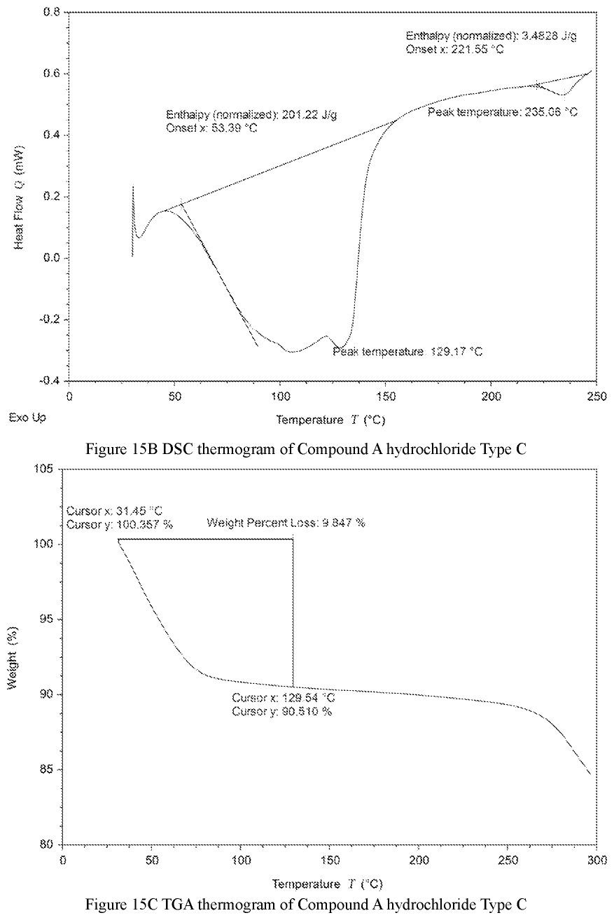

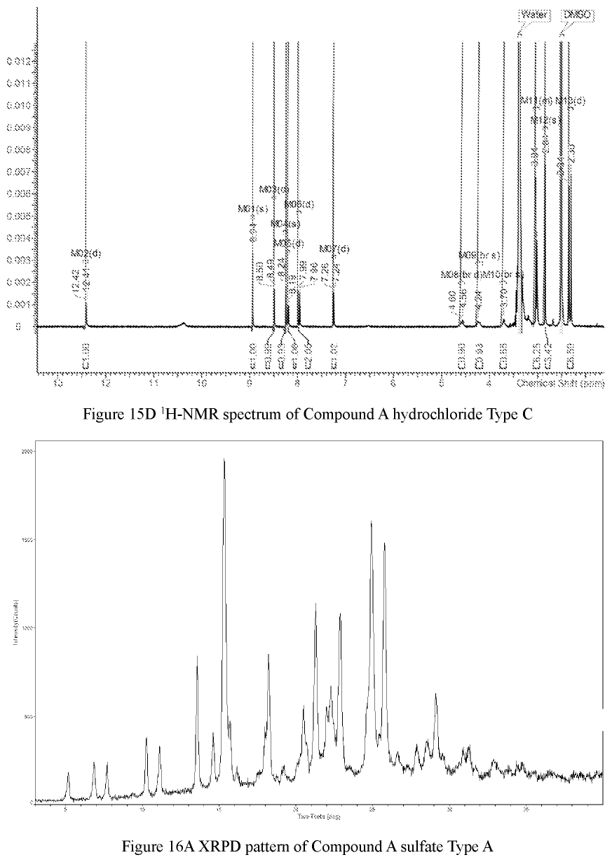

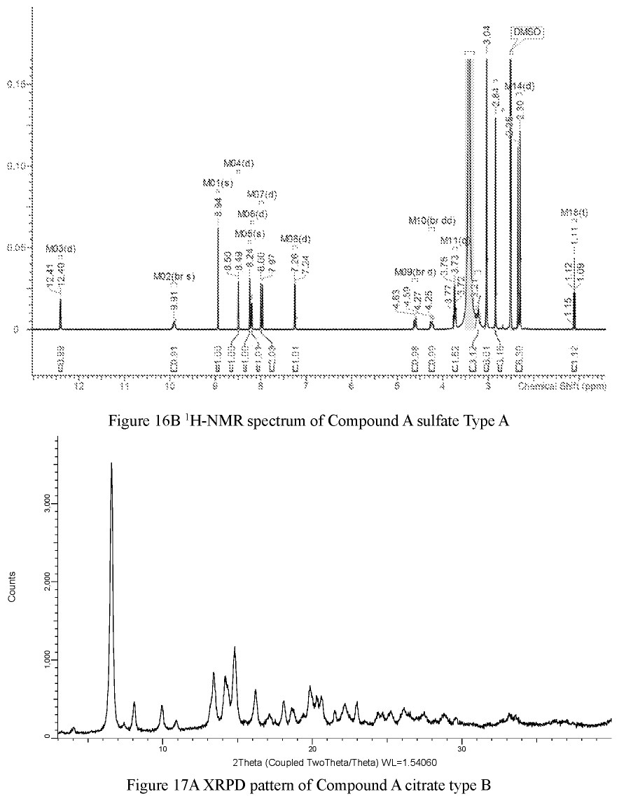

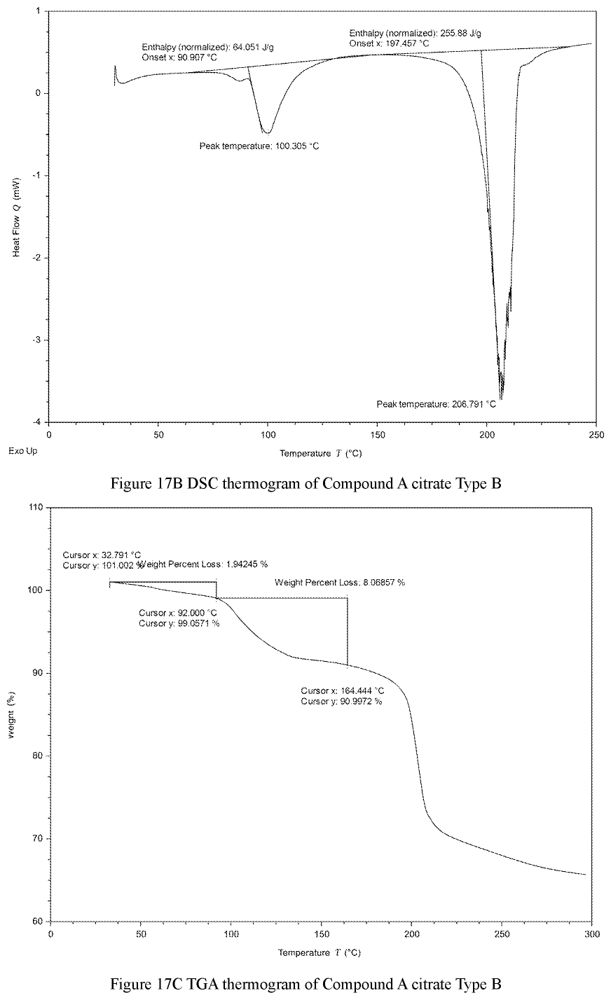

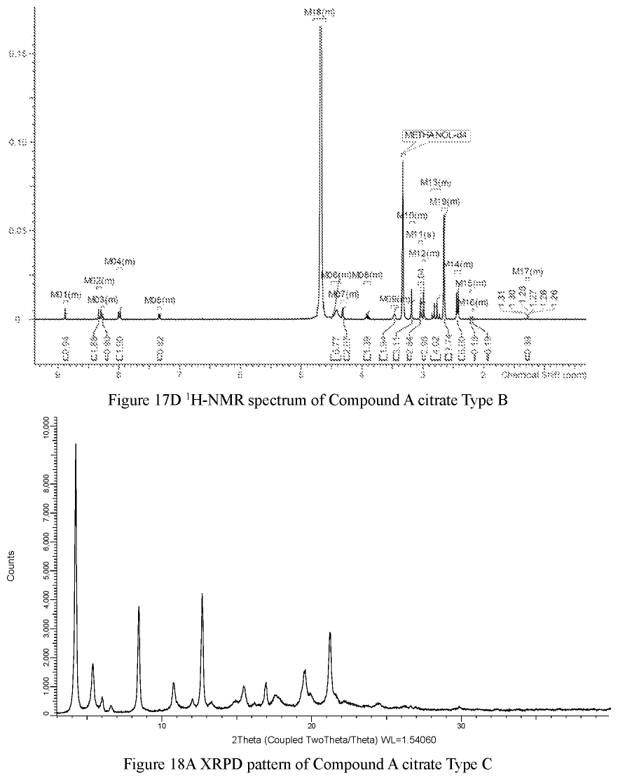

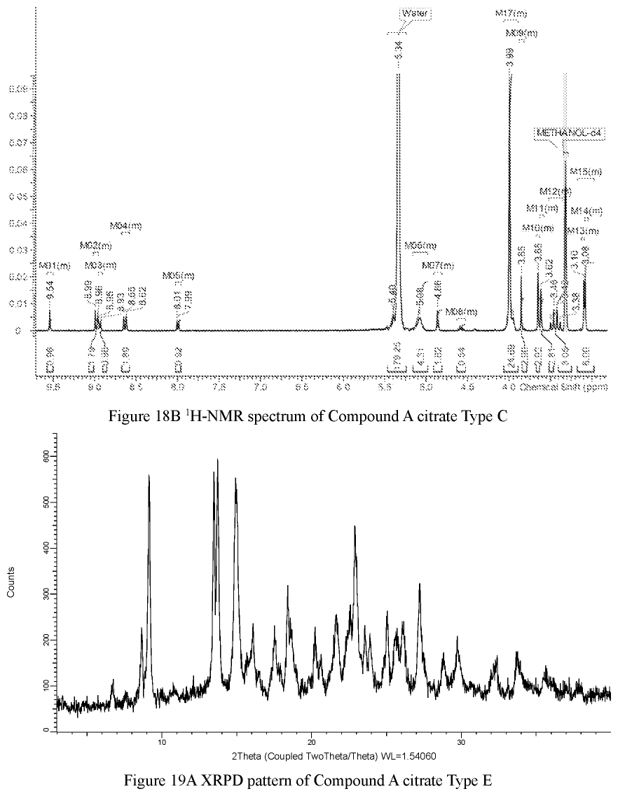

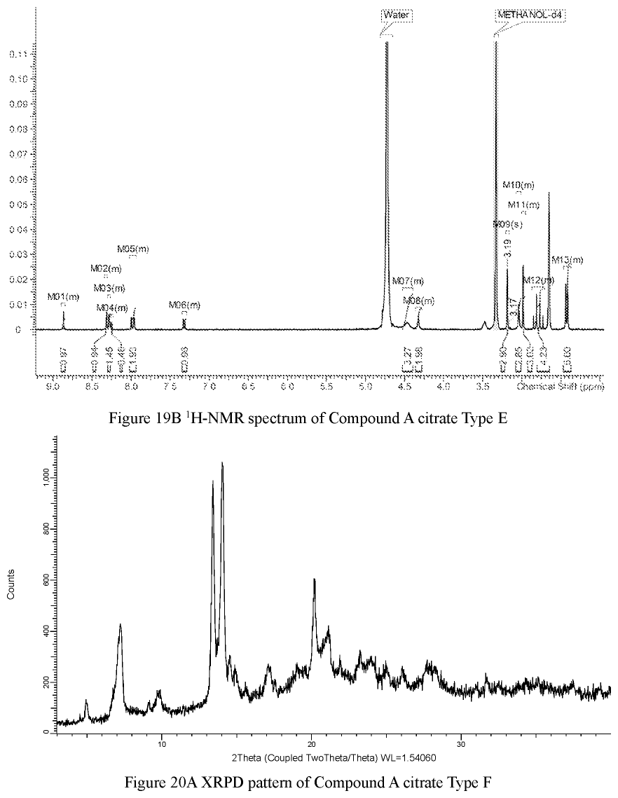

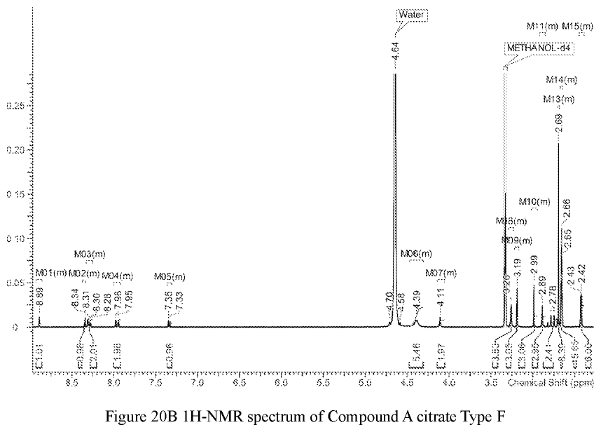

26. The crystalline form of any one of claims 14-25, substantially characterized by a powder X-ray diffraction pattern selected from the group consisting of FIG. 1A, FIG. 2A, FIG. 3A, FIG. 4A, FIG. 5A, FIG. 7A, FIG. 8A, FIG. 9A, FIG. 10A, FIG. 11A, FIG. 12A, FIG. 13A, FIG. 14A, FIG. 15A, FIG. 16A, FIG. 17A, FIG. 18A, FIG. 19A or FIG. 20A.

27. A pharmaceutical composition comprising a therapeutically effective amount of crystalline form according to any one of claims 14-26, and optionally one or more pharmaceutically acceptable carrier(s).

28. A method for treating or preventing a disorder or a disease selected from inflammatory disorder, autoimmune disease, or a cancer, comprising administering a subject in need thereof a therapeutically effective amount of the crystalline form according to any one of claim 14-26, or the pharmaceutical composition of claim 27.
